(19) United States
(12) Patent Application Publication
(10) Pub. No.: US 2006/0281754 A1
(43) Pub. Date: Dec. 14, 2006

(54) Method of treating liver disease

(76) Inventors: Jude A. Oben, London (GB); Anna M. Diehl, Ellicott City, MD (US)

Correspondence Address: STITES & HARBISON, PLLC, 400 W MARKET ST, SUITE 1800, LOUISVILLE, KY, 40202-3352, US

(21) Appl. No.: 10/550,919

(22) PCT Filed: Mar. 24, 2004
(86) PCT No.: PCT/GB04/01323

§ 371 Date: Sep. 1, 2006

Related U.S. Application Data
(60) Provisional application No. 60/458,450, filed on Mar. 28, 2003.
(60) Provisional application No. 60/466,646, filed on Apr. 30, 2003.
(60) Provisional application No. 60/493,559, filed on Aug. 8, 2003.
(60) Provisional application No. 60/503,142, filed on Sep. 12, 2003.

(51) Int. Cl. A61K 31/517; A61K 31/138

(52) U.S. Cl. 514/252.17; 514/651

(57) ABSTRACT

Treating diseased or damaged tissue, particularly the liver, using stem cells. The hepatic stem cell population of a subject suffering from diseased or damaged tissue can be expanded by administering at least one regulator of the sympathetic nervous system. The regulator can be an adrenoceptor agonist or antagonist, adrenoceptor antagonists, prazocin, being particularly preferred. The invention also includes the use of agents which mobilize stem cells in the manufacture of medicaments for the treatment of diseased or damaged tissue.

### CROSS REFERENCE TO RELATED APPLICATIONS

This application claims benefit of U.S. Provisional Application Nos. 60/458,450, filed Mar. 28, 2003, 60/466,646, filed on Apr. 30, 2003 and 60/493,559, filed on Aug. 8, 2003 (which is hereby incorporated by reference).

This invention concerns the treatment of diseased or damaged tissue in particular using stem cells, and especially using stem cells mobilized

The following abbreviations are used herein:
sympathetic nervous system (SNS), prazosin (PRZ), 6-hydroxydopamine (6-OHDA), hepatic progenitor cell (HPC), autonomic nervous system (ANS), norepinephrine (NE), natural killer T (NK-T) cells, half methionine-choline deficient plus ethionine (HMCDE), control methionine choline diet (CMCD), stem cell factor (SCF), interleukin (IL), leukaemia inhibitory factor (LIF), granulocyte-macrophage colony stimulating factor (GM-CSF), ganulocyte colony stimulating factor (G-CSF), vascular endothelial growth factor (VEGF), hepatocyte growth factor (HGF), cytokeratin (CK), dopamine Ohydroxylase (Dbh), extracellular signal-regulated kinase (ERK), isoprenaline (ISO), M2pyruvate kinase (MPK), mitogen-activated proteinkinase/extracellular signal-regulated kinase kinase (MEK), mouse hepatic oval cells (HOC), and propanalol (PRL).

Recovery from liver damage might be enhanced by encouraging repopulation of the liver by endogenous hepatic progenitor cells. Bone marrow-derived progenitors may differentiate into oval cells—resident hepatic stem cells that promote liver regeneration and repair. Little is known about the mediators that regulate the homing or accumulation of these cells in the liver. The sympathetic nervous system (SNS) innervates bone marrow, and adrenergic inhibition mobilizes hematopoeitic precursors into the circulation. Thus, we hypothesized that SNS inhibition would promote hepatic accumulation of progenitor cells and reduce liver damage in mice fed anti-oxidant depleted diets to induce liver injury. Our results confirm this hypothesis. Compared to control mice that were fed only the anti-oxidatit depleted diets, mice fed the same diets with Prazosin (PRZ, an alpha-1 adrenoceptor antagonist) or 6-hydroxydopamine (6OHDA, an agent that induces chemical sympathectomy), had significantly increased numbers both of oval cells and putative bone marrow-derived hepatic progenitors. Increased hepatic progenitor cell accumulation was accompanied by less hepatic necrosis and steatosis, lower serum aminotransferases, and greater liver and whole body weights. Neither PRZ nor 6-OHDA affected the expression of cytokines, growth factors or growth factor receptors that are known to regulate progenitor cells. In conclusion, stress-related sympathetic activity modulates progenitor cell accumulation in damaged livers and SNS blockade with alpha-adrenoceptor antagonists enhances hepatic progenitor cell accumulation and improves recovery from liver damage.

The liver's progenitor cell compartment is activated if the resident mature hepatocytes reach a critically low number, such as after severe hepatic injury, or if the mature hepatocytes are prevented from dividing by hepatotoxic drugs. One hepatic progenitor cell (HPC) compartment, the oval cells, are resident within the liver's canals of Herring—the terminal branches of the biliary tree. The source of oval cells themselves is debated but there is some evidence that they may be derived from pluripotent progenitors that reside in the bone marrow. The factors involved in expanding hepatic progenitor cell populations within the liver are not well understood. The identification of such factors is an important therapeutic goal because they may be useful to support patients with acute liver failure until a suitable organ is found for transplant. Indeed, if successful, targeted expansion of endogenous HPC may even obviate the need for orthotopic liver transplantation.

Emerging evidence suggests that the autonomic nervous system (ANS) may regulate the accumulation of HPC in the liver. The parasympathetic nervous system appears to promote this process because vagotomy reduces the expansion of HPC numbers in rats with drug induced hepatitis. Similarly, after transplantation (which surgically denervates the liver), human livers that develop hepatitis have fewer HPC than native, fully innervated livers, with similar degrees of liver injury. The decreased accumulation of HPC in transplanted livers may alter their regenerative response to injury because the rate of fibrosis is often accelerated in liver transplant recipients with chronic hepatitis.

Although the sympathetic nervous system (SNS) is known to modulate both liver regeneration and hepatic fibrogenesis, it is not known if these effects reflect the ability of the SNS to influence HPC accumulation in injured livers. Thus, the aim of the present study was to test the hypothesis that the SNS affects the expansion of HPC. We used established models of HPC accumulation involving administration of anti-oxidant depleted diets plus ethionine to cause liver injury and inhibit mature hepatocyte replication. We then manipulated the SNS by adrenoceptor antagonism with prazosin (PRZ) or chemical sympathectomy with 6-hydroxydopamine (6-OHDA), in order to reduce the activity or production of the SNS neurotransmitter, norepinephrine (NE). HPC numbers in control and SNS-inhibited livers were analysed by both flow cytometry and immunohistochemistry. Because the SNS is known to promote the hepatic accumulation of natural killer T (NK-T) cells, liver NK-T cells were evaluated concurrently to monitor the physiological efficacy of SNS inhibition. Our results demonstrate that SNS inhibition significantly enhances the accumulation of BPC and reduces liver injury. This suggests that adrenoreceptor blockade might be used therapeutically to expand HPC and promote liver regeneration in circumstances that prevent the replication of mature hepatocytes.

According to the present invention there is provided a method of treating diseased or damaged tissue which comprises administering an agent for mobilizing stern cells.

The invention further provides the use of an agent for the mobilization of stem cells in the manufacture of a medicament for the treatment of diseased or damaged tissue.

C57BL-6 mice, 10-18 weeks old, were from Jackson Laboratory (Bar Harbor, Me.) were subjected to the following experiments.

The diet was a modification of the half-choline deficient diet CICN, Aurora, Ohio) that has been shown to cause hepatic accumulation of HPC within 2 weeks. In addition to choline deficiency the diet used here was also 50% deficient in methionine to enhance oxidative injury to the liver, This diet was administered with ethionine (0.15%) in drinking water (Hepatology 2001; 34; 519-522) and the combination treatment is hereafter referred to as half methionine choline deficient diet plus ethionine (HMCDE). The control methionine choline diet (CMCD) was also from ICN Prazosin (PRZ) and DL-Ethionine (E) were from Sigma, St Louis, Mo.).

Chemical sympathectomy was achieved by intra-peritoneal (IP) injection of 6-hydroxydopamine (6-OHDA), 100 mg/kg for 5 consecutive days as described in Ann Surg 2001; 233:266-275. Thereafter, 6-OHDA was administered at 100 mg/kg i.p., three times per week to ensure continued sympathectomy. The dose and dosing regimen for 6-OHDA has been previously shown to virtually deplete norepinephrine in rodent tissues (Hepatology 2002; 35:325-331).

Mice were divided into 4 groups, with each group containing 10 to 12 animals. Group I—Control diet; Group 2—HMCDE plus saline i.p.; Group 3—HMCDE plus prazosin in drinking water; Group 4—HMCDE plus 6-OHDA i.p. Experiments were performed on 2 separate occasions. Therefore, final results are derived from ˜100 mice (10-12 mice/group/experiment×2 experiments).

All mice were weighed at the beginning of the feeding period and weekly thereafter until killed. At the time of sacrifice, sera were collected from all the animals in each group and liver tissue from half the animals in each group. Collected liver tissues were either fixed in buffered formalin, preserved in OCT compound (Sakura, Torrance, Calif.) and processed for histology or snap frozen in liquid nitrogen and stored at −80° C. until RNA was isolated, The livers from the remaining animals in each group were prepared for flow cytometry as described below.

Wedges of liver from each of the mice were prepared for histology and immunochemistry as described previously (Am J Pathol 2002: 161:5210530 and Nat. Med.2000; 6: 998-1003). For histology, tissues were formalin fixed, paraffin embedded and 5-μm sections were stained with hematoxylin and eosin (H&E). Coded samples were examined by an experienced liver pathologist who was blinded to treatment groups. Hepatocellular fat accumulation was scored as, no fat 0, focal fat accumulation in <1% of the hepatocytes=F, fat in 1-30% of the hepatocytes 1+, fat in 31-60% of the hepatocytes=2+, and fat in 61-100/o of the hepatocytes 3+. To evaluate the amount of hepatocyte necrosis, the number of necrotic hepatocytes was counted in 10 randomly selected fields with a 20× lens.

Immunohistochemical analysis of SPC was performed with a mouse monoclonal OV6-type antibody (from Dr Stuart Sell, Albany Medical College, Albany, N.Y.) reacting with cytokeratins 14 and 19; a rabbit polyclonal antibody against 56 and 64 kD human callus cytokeratins (Dako, Denmark) and a rat monoclonal antibody to cytokeratin 19 as described Am J Pathol 2002;161:521-530.

Details of the staining procedures are as described Am J Pathol 2002;161:521-530. Briefly, 4 μm thick paraffin sections were deparaffinized and rehydrated, followed by heating in a microwave oven for 10 minutes at 750 Watt in citrate buffer, pH 6.0. Incubation with the primary antibodies was performed at room temperature for 30 minutes. Mouse monoclonal OV6 antibody and rat anti-cytokeratin 19 were detected using the DAKO Animal Research Kit, peroxidase (Dako, Denmark). The rabbit polyclonal antibody against 56 and 64 kD human callus cytokeratins was detected by anti-rabbit Envision (Dako, Denmark) as described in J Pathol 2003; 199: 191-200.

HPC were defined as small cells with an oval nucleus and little cytoplasm. These cells occur either singularly or organized in arborizing, ductular structures. They have strong reactivity for liver type cytokeratins, OV-6 and bile duct type cytokeratin 19.

To evaluate the effect of treatments on the HPC compartment, coded samples were examined by an experienced liver pathologist blinded to treatment groups. For each liver section, the number of HPC in 5, randomly selected, non-overlapping, high power (×40 objective) fields was counted. Interlobular bile ducts, were defined as bile ducts with a lumen, associated with a branch of the hepatic artery. Interlobular bile ducts were not considered progenitor cells and, thus, were not counted as such.

The presence of alpha-1 adrenergic receptors on HPC was detected on frozen sections using a rabbit polyclonal anti-alpha 1 adrenergic receptor antibody (sc10721, Santa Cruz Biotech, Santa Cruz, Calif., dilution 1/20), followed by undiluted anti-rabbit Envision (Dako, Denmark). For immunofluorescence studies, the anti-alpha-1 adrenergic receptor antibody was combined with a polyclonal antibody against 56 and 64 kDa human callus cytokeratins (Dako, Denmark; dilution 1:100). The primary antibodies were applied sequentially and subsequently detected with swine-antirabbit FITC or TRITC conjugates. In controls sections primary antibodies were omitted. All stainings were performed on 4 representative sections.

Sera from all the animals were analysed for alanine aminotransferase (ALT) activity by the Clinical Chemistry Laboratory of the Johns Hopkins Hospital.

Total RNA was isolated from frozen liver samples according to the method of Chomczynski and Sacchi as described in Gastroenterology 2002; 123: 1304-1310. RNA concentration was determined by optical density and quality was assessed by agarose gel electrophoresis and ethidium bromide staining. Commercial ribonuclease protection assay (RPA) kits with probes for murine cytokines (PharMingen, San Diego, Calif.) were used to evaluate factors that might be involved in the recruitment and expansion of HPC after liver injury. The factors studied were Stem Cell Factor (SCF), Hepatocyte Growth Factor (HGF), Interleukin-7 (IL-7), IL-11, Leukaemia Inhibitory Factor (LIF), Granulocyte-Macrophage Colony Stimulating Factor (GM-CSF), Granulocyte Colony, Stimulating Factor (G-CSF), Vascular Endothelial Growth Factor (VEGF), and its receptors, VEGFR1 and VEGFR3. Details of the RPA are described in J Pathol 2003; 199: 191-200.

The hepatic non-parenchymal cell fraction, which contains the oval cell population and the NK-T cell populations, were isolated as described in Hepatology 1998; 27: 433455 and Gastroenterology 2002; 123:1304-1310. Briefly, livers were carefully removed and homogenized in Stomacher80 (Seawood, England). The homogenate was then passed through a 100-μm wire mesh and liver cells were collected by centrifugation at 450 g. Mononuclear cells were purified from this fraction by centrifugation at 900 g over 35% Percoll gradients (Amersham Pharmacia Biotech) and incubated with normal mouse serum (Sigma, St Louis, Mo.) and Fc-receptor block (anti-CD16/CD32) to prevent non-specific binding, plus APC-conjugated anti-mouse Thy-1.2 (the C57BL-6 form of the Thy-1 antibody) and antibodies directed against hematopoeitic lineage markers (LIN, a mix of anti-mouse CD4, CD8, CD3, CD45, CD19, Mac-1, Gr-1, Ter119). For NK-T cell labeling, the mononuclear cells were incubated with FITC-conjugated anti-mouse NK-1.1 and PE-conjugated anti-mouse CD3. All antibodies were from Pharmingen except anti-mouse Ter119, which was from Cedarline lab, Canada. After incubation, pellets were washed to remove unbound antibodies, fixed with 2% formaldehyde and evaluated by FACS (Becton Dickenson). As described in Science 1999; 284:1168-1170, LIN^{-ve}/Thy-1^{+ve} cells, were classified as putative bone marrow-derived, hepatic progenitor cells. Data was analyzed by Cell Quest software Wecton Dickenson) to determine changes in these cell populations in different treatment groups.

All values are expressed as mean±SEM. The group means were compared by unpaired t-test or ANOVA using Graphpad Prism 3.03 (San Diego, Calif.).

To determine the gross effects of the diets on our experimental animals, the weights of the animals at the start and end of the experiments were compared. Mice fed the control diet gained a mean of 3 g (12% of starting body weight) during the course of the study.

In contrast, mice fed the HMCDE diet lost a mean of 3 g (12% of starting body weight). Mice fed the HMCDE diet in the presence of PRZ or 6-OHDA, however, only lost a mean of 2 g (7% and 8% of starting body weight). Therefore, SNS inhibition slightly, but significantly, attenuates the weight loss that occurs during consumption of antioxidant-depleted diets.

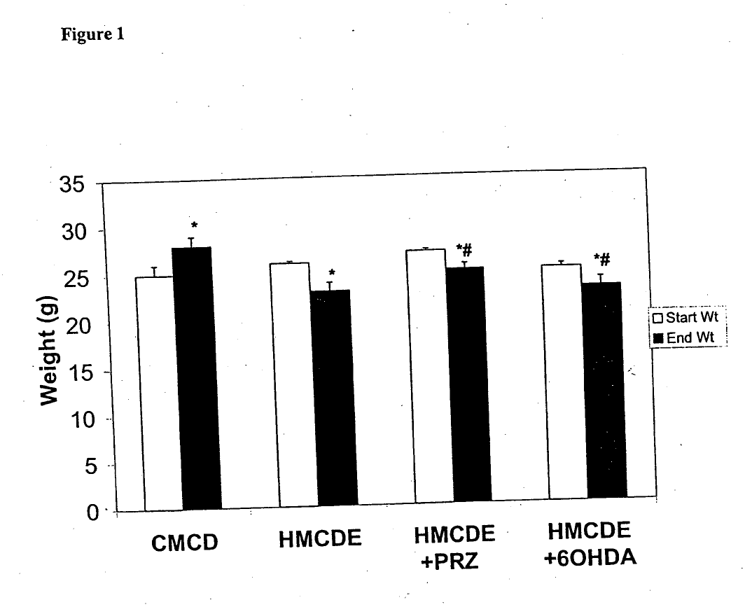

FIG. 1 shows the effect of control and antioxidant-depleted diets on body weight. Mean±SEM body weights of mice before and after 4 weeks of feeding. Only mice fed the control diet (CMCD) gained weight (*P<0.04 vs baseline); all groups that were fed half methionine choline deficient diets (HMCDE) lost weight (*P<0.001 for post-versus pre-HMCDE, P<0.008 for post-versus pre-HMCDE+PRZ, P<0.03 for post-versus pre-HMCDE+6-OHDA). However, HMCDE+PRZ and HMCDE+6-OHDA groups lost less weight than the HMCDE group (*P<0.05).

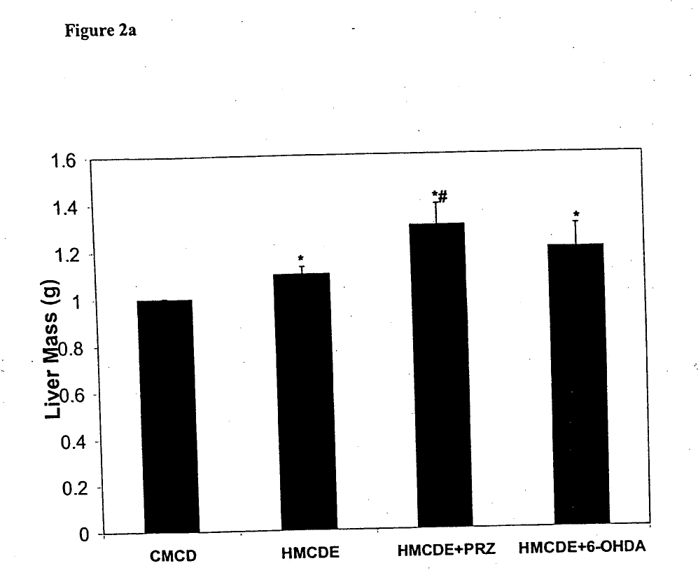

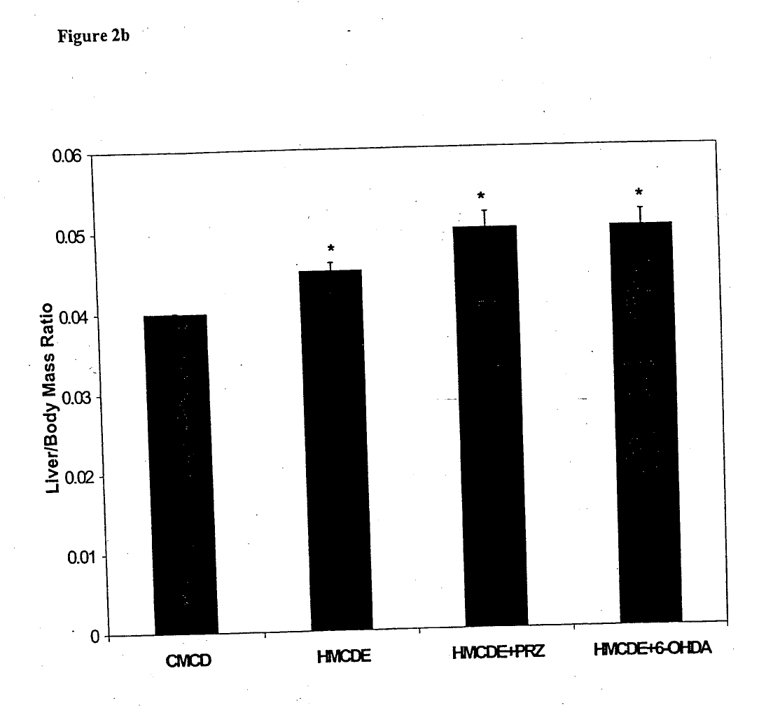

The treatments also influenced liver mass, the effect of SNS inhibition on liver mass in mice with diet-induced liver damage being shown with reference to FIGS. 2a, b.

FIG. 2a shows that compared to mice fed control diets (CMCD), absolute liver mass was greater in all groups fed HMCDE diets (*P<0.01). Absolute liver mass in the HMCDE+PRZ group was greater than the group fed HMCDE alone (#P<0.04). FIG. 2b shows liver/body weight ratios also increased on HMCDE diets (*P<0.02 for all groups versus CMCD) and tended to be greater in HMCDE-treated mice that received SNS inhibitors, although the difference between these groups and those fed HMCDE diets alone did not achieve statistical significance.

In mice with an intact SNS, as well as in those treated with SNS inhibitors, the HMCDE diet caused an increase in liver mass (FIG. 2a), as well as liver/body mass ratio (FIG. 2b) above that of the control diet. Increases in both parameters tended to be greater in mice that were treated with SNS inhibitors, but the differences in liver mass achieved statistical significance only for the HMCDE+PRZ treated group. Thus, although SNS inhibition reduced diet-related loss of body mass, it tended to enhance diet-induced hepatomegaly.

Liver histology confirms that, as expected, HMCDE diets caused hepatic steatosis and necrosis.

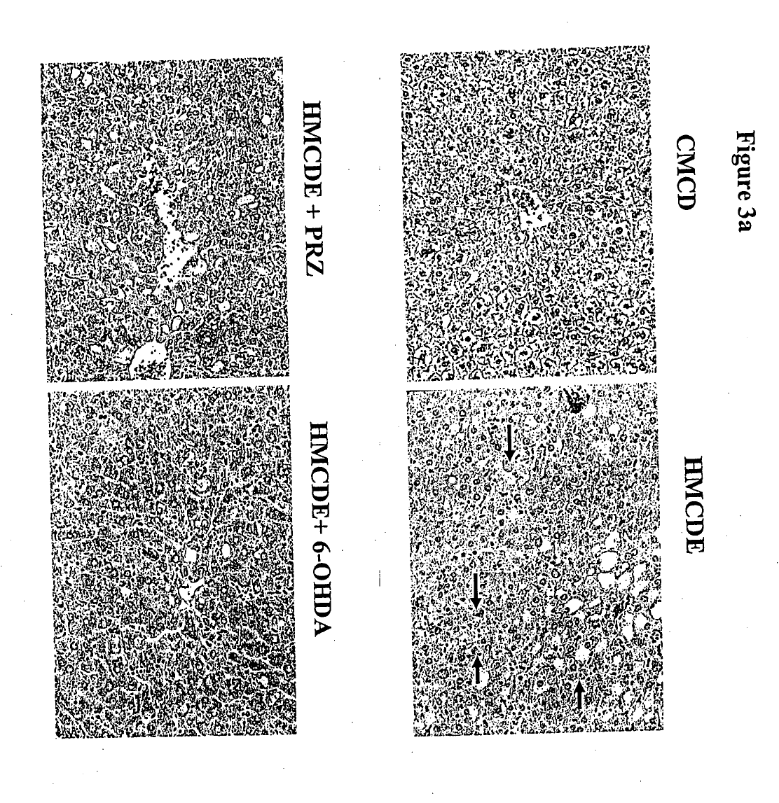

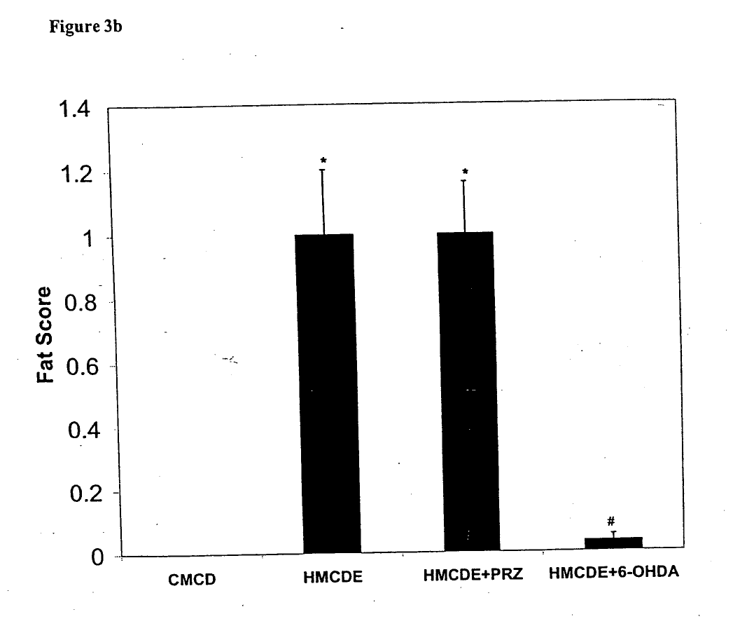

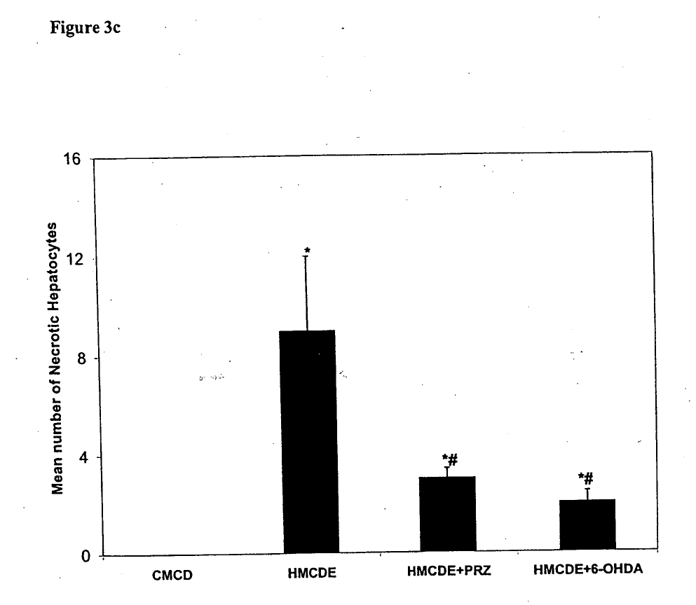

FIGS. 3a-c show the effect of SNS inhibition on diet-induced liver injury.

FIG. 3a shows liver histology, images having been captured with a 25× lens. Hematoxylin and eosin stained sections of representative mice that were fed control diet (CMCD) (top left) showed no fat accumulation or necrosis. A section from a representative HMCDE fed animal showed 2+ fat accumulation and areas of hepatocyte death—arrowed (top right), while one from a HMCDE+PRZ fed mouse showed 1+ fat accumulation and reduced liver cell death (bottom left). The liver section from a representative HMCDE+6-OHDA fed animal showed focal (F+) fat accumulation and minimal necrosis (bottom right).

FIG. 3b shows fat scores comparing mice fed control diets (CMCD), the HMCDE and HMCDE+PRZ groups had more fat (*P<0.0004). The HMCDE+6-OHDA treated group had significantly less fat than the HMCDE alone group (#p<0.0001).

FIG. 3c shows necrosis scores. Compared to controls (CMCD), all HMCD-fed groups had more necrotic hepatocytes (*P<0.01), but compared to mice that were fed the HMCDE diet alone, the numbers of necrotic hepatocytes were reduced in HMCDE+PRZ (#P<0.05) or HMCDE+6-OHDA ( P<0.05).

FIG. 3d shows serum alanine aminotransferase (ALT) levels, a marker of liver injury, were increased in all HMCDE-fed groups compared to CMCD controls (*P<0.01). Compared to HMCD-fed mice, mice treated with HMCDE+PRZ or HMCDE+6-OHDA bad lower ALT levels ( P<0.03).

Histologic evidence of liver injury was accompanied by significant increases in serum ALT values (FIG. 3d). Treatment with 6-OHDA, but not PRZ, significantly reduced the fat score (FIG. 3b). However, both SNS inhibitors significantly reduced hepatic necrosis (FIG. 3c) and serum ALT values (FIG. 3d), These findings demonstrate that PRZ and 6-OHDA-related increases in liver mass occured despite improvements in hepatic steatosis (6-OHDA) and/or necrosis (PRZ and 6-OHDA) and suggest that SNS inhibition might improve liver regeneration.

Diet induced liver injury itself elicits a compensatory regenerative response, as evidenced by the accumulation of HPC in control mice that were fed the HMCDE diet.

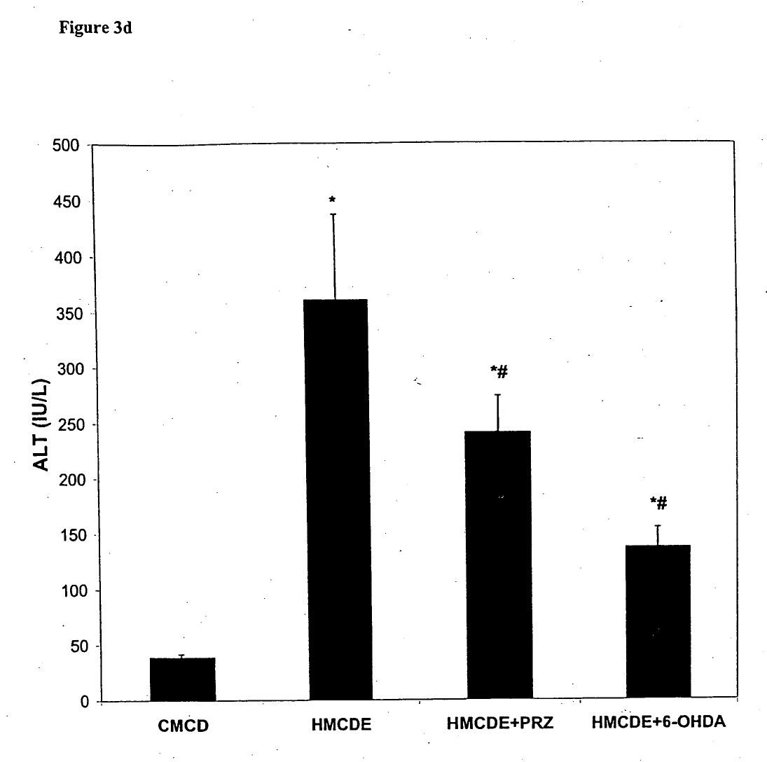

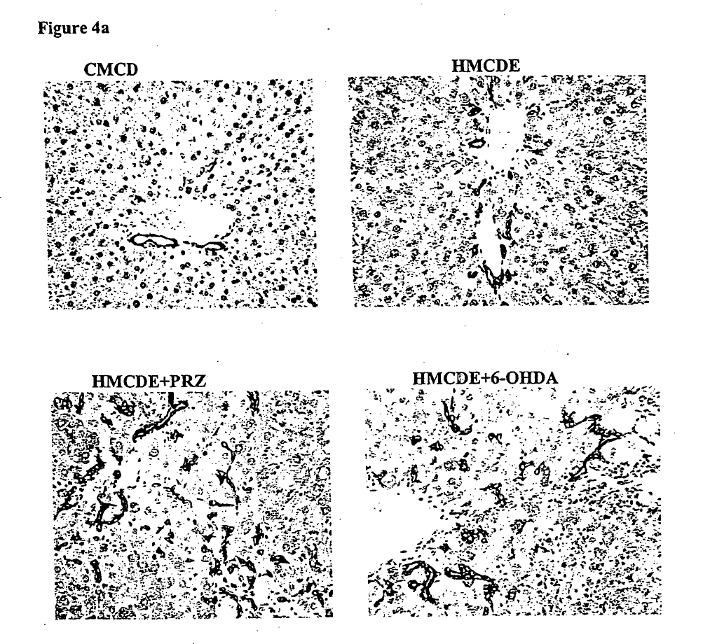

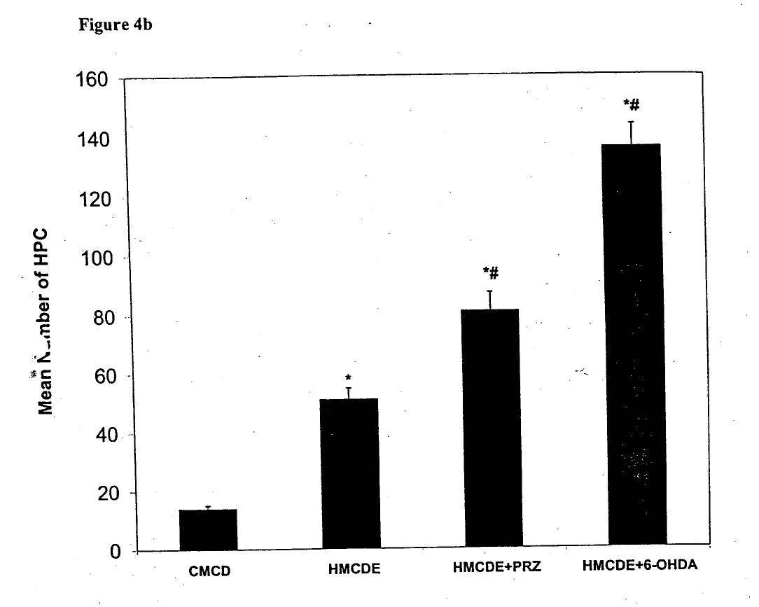

FIG. 4 shows the effect of SNS inhibition on the numbers of hepatic progenitors in liver with diet-induced damage.

FIG. 4a shows immunohistochemistry for oval cells, in representative mice that were fed control diet (CMCD) (top left), HMCDE (top right), HMCDE diet+PRZ (bottom left) or HMCDE+6-OHDA (bottom tight). Oval cells were stained brown.

FIG. 4b shows that the numbers of oval cells increased in all HMCD-fed groups compared to CMCD controls (*P<0.0001). Both groups treated with SNS inhibitors had more oval cells than mice that were fed HMCDE diets alone (#P<0.001).

FIG. 4d shows that when putative bone marrow-derived hepatic progenitors (i.e., LIN^{-ve}/Thy-1^{+ve} are quantified by flow cytometry, livers from groups treated with HMCDE+PRZ or HMCDB+6-OHDA contain more of these cells than CMCD controls (*P<0.01), although HMCDE feeding alone did not expand this compartment. Compared to mice fed HMCDB diets alone, mice fed HMCDE+PRZ or HMCDE+6-OHDA had more LIN^{-ve}/Thy-1^{+ve} cells (#P<0.03 and <0.05, respectively).

The increased HPC were demonstrated immunohistochemically by an increase in the numbers of bile duct type cytokeratin—positive oval cells (FIG. 4a, b) and by flow cytometry quantification of bone marrow lineage marker negative (LIN 8−) cells that expressed Thy 1.2 (FIG. 4c). SNS inhibition with either PRZ or 6-OHDA significantly augments diet-induced HPC expansion by both assays (FIG. 4a-c). The hepatic accumulation of HPC is a fairly specific consequence of SNS inhibition because, as expected, the numbers of NK-T cells in the livers of HMCDE-treated mice (8±1% liver mononuclear cells) decrease significantly after treatment with either PRZ (3.5±0.5%, P<0.05) or 6-OHDA (3.6±0.6%, P<0.05). Given that SNS inhibition also reduces HMCDE-induced liver injury (FIG. 3) and stabilizes body weight (FIG. 1), it seems unlikely that SNS inhibition generates a greater requirement for hepatic HPC accumulation. Rather, these findings suggest to us that BPC expansion might contribute to the hepatoprotective effects of SNS inhibition.

Other groups have shown that the hepatocyte mitogen, hepatocyte growth factor (HGF), induces oval cell proliferation, promotes liver regeneration and protects the liver from hepatotoxicity. Given the similarities between the effects of SNS inhibition and HGF, it was important to determine if SNS inhibition increased hepatic HGF expression. Consistent with other reports that liver injury induces compensatory expression of HGF and other factors that promote regeneration, we found that treatment with HMCDE increased the hepatic expression of HGF more than 2 fold above control (P<0.04 versus CMCD)—data not shown. However, SNS inhibition with PRZ or 6-OHDA did not augment this response. Therefore, the hepatoprotective effects of SNS inhibition are not easily explained by HGF induction, although our studies do not exclude the possibility that SNS inhibition sensitizes the liver to HGF actions.

Oval cells and bone marrow-derived hepatic progenitors express c-kit, the receptor for stem cell factor (SCF). Other cytokines, such as interleukin (IL)7 and LIF, may also promote progenitor cell accumulation in injured tissues because after cardiac injury, these factors help to recruit bone marrow-derived stem cells to the injured heart. IL-6 is expressed by bone marrow derived cells in regenerating livers and this cytokine has an important hepatoprotective effect because mice that are genetically deficient in IL-6 exhibit inhibited liver regeneration after partial hepatectomy. Other cytokines, such as G-CSF, that signal through gp-130 receptors may be able to compensate for IL=6 deficiency and promote regeneration when the latter cytokine is deficient. Vascular endothelial growth factor (VEGF) may also play some role in the expansion of HPC because it is a growth factor for hematopoietic stem cells, which express VEGF receptors. To begin to clarify the mechanisms by which SNS inhibition enhances HPC accumulation in injured livers, we evaluated the effects of SNS inhibition on the hepatic expression of G-CSF, GM-CSF, IL-6, IL-7, IL-11, LIF, SCF, VEGF and its receptors VEGFR1 and 3. RPA of whole liver RNA was used to compare the expression of these factors in control (CMCD) mice and nice treated with HMCDE plus or minus SNS inhibitors. No appreciable GM-CSF, IL-6, IL-7, IL-11, SCF or LIF expression could be demonstrated by this assay (data not shown). HMCDE-treatment, however, increased G-CSF expression about 2 fold, regardless of SNS inhibition (P<0.05 all HMCDE groups versus CMCD). VEGF and its receptors were expressed in both control and all HMCDE-treated mice, but SNS inhibition did not alter the expression of these factors (data not shown). Thus, although these experiments do not exclude the possibility that the expression of one or more of these factors may have changed in some small population of liver cells after SNS inhibition, these progenitor cell trophic factors do not appear to be the major targets for SNS regulation.

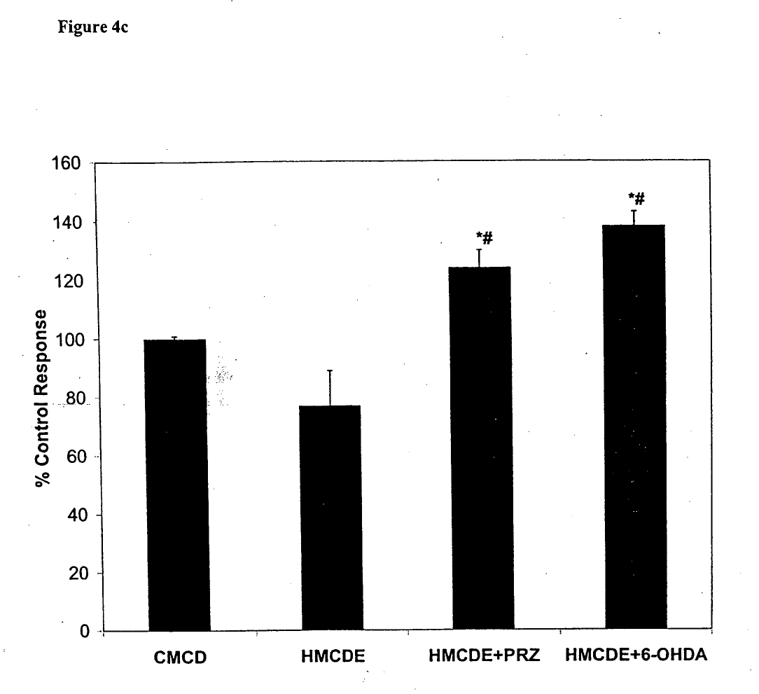

To determine if the effects of SNS inhibition on the BPC compartment might be mediated via direct interaction between NE and adrenoceptors on BPC, we used immunohistochemistry to determine if IPC express alpha-1 adrenoceptors. Our results show that bile duct type cytokeratin-positive oval cells do express apha-1 adrenoceptors (FIG. 5a,b). Therefore, direct regulation of this HPC compartment by NE is plausible.

FIG. 5a shows immunohistochemistry for alpha-1 adrenoceptors on bile duct type cytokeratin-positive oval cells in a liver section from a representative mouse fed HMCDE. Oval cells expressing alpha-1 adrenoceptors were stained brown.

FIG. 5b shows that immunofluorescence studies confirm the co-localisation of alpha-1 adrenoceptors on bile duct type cytokentin-positive oval cells. Without the primary antibodies, binding of the secondary antibodies was negligible (not shown). Alpha-1 adrenoceptors—red, cytokeratins—green, co-localization—yellow.

The sympathetic nervous system (SNS) nerve terminals contain both norepinephrine (NE) plus NPY and other molecules. Prazosin blocks only the alpha-1 adrenoceptor mediated effects of NE. 6-OHDA, however, depletes the SNS nerve terminals of NPY and NE. Therefore, that a larger number of oval cells and bone marrow derived progenitor cells are seen with 6-OHDA treatment suggests that NPY is inhibitory and that removing NPY removes this inhibition and leads to larger numbers of liver stem cells.

Critical shortages of donor livers for orthotopic liver transplantation have become a major limiting factor in efforts to reduce mortality of patients with end-stage liver disease. Therefore, alternative strategies to replace severely damaged livers must be developed. Studies in mice with massive toxin-induced liver injury have demonstrated that liver cell transplantation can effectively regenerate the liver. Hence, many groups are working to optimize cell transplantation strategies. An alternative, but complementary, approach that might be used to enhance regeneration of injured livers involves treatment to encourage repopulation of the liver by endogenous hepatic progenitors. The general feasibility of this strategy is supported by recent evidence that the administration of cytokine mixtures to mobilize native, bone marrow-derived progenitor cells heals experimentally-induced myocardial infarcts in mice. However, although it has been observed hitherto that certain bone marrow cells can differentiate into oval cells and mature hepatocytes, the relative importance of bone marrow-derived progenitors, as opposed to resident hepatic progenitors (i.e. oval cells) and mature hepatocytes for liver regeneration remains uncertain. Moreover, even if certain progenitor cell populations do contribute to recovery from liver injury, little is known about the mediators that regulate their accumulation within ;the liver. Therefore, the identification of these factors is an important first step in the development of treatments that seek to expand hepatic progenitor cell populations.

Presumably, endogenously produced factors that induce the hepatic accumulation of liver progenitor cells are increased, to some extent, during liver damage because regenerative responses have been observed hitherto in most injured livers. However, other factors that increase during injury might inhibit progenitor cell expansion and this would compromise reconstruction of a healthy organ, if the progenitors play a role in liver regeneration. Thus, one way to enhance recovery from liver injury might be to neutralize the actions of endogenous factors that limit the expansion of native HPC populations. To explore the validity of this concept, we studied mice that were treated with half strength, methionineicholine deficient diets supplemented with ethionine (HMCDE), because this murine model of liver injury is known to increase hepatic oval cells. Our results show that stress-related SNS activity is one of the endogenous factors that modulate UPC accumulation in damaged livers. However, the mechanisms for this remain uncertain because we found no effect of SNS inhibition on several factors that are thought to promote progenitor cell accumulations.

On the other hand, at least one mechanism that regulates the accumulation of oval cells in the livers of choline deficient mice has been reported. For example, it has been shown hitherto that TNF-α increases in mice that are fed choline-deficient diets and demonstrated that proliferating hepatic oval cells produce this cytokine. Moreover, it was observed that TNF-α is required for oval cell expansion because this response is abrogated by genetic disruption of TNFR1. These observations are particularly intriguing because TNF-α and TNFR1 has been shown by other workers to be necessary for liver regeneration after partial hepatic resection and other types of liver injury. Although we did not evaluate potential interactions between TNF-α and the SNS in our model, work in many other systems demonstrates clear evidence for cross talk between signaling mechanisms that are activated by TNF-α and those that are modulated by sympathetic neurotransmitters, such as NE.

At the very least, these interactions may explain our observation that PRZ and 6-OHDA reduced HMCDE-induced liver injury, because NE inhibits cytokine inducible nitric oxide (NO) production in hepatocytes and NO protects hepatocytes from TNF-toxicity. Thus, NE promotes TNF-α-mediated hepatotoxicity and agents that block NE generally inhibit this. Whether or not NE-TNF-α interactions influence HPC expansion has not been investigated, but merits evaluation because it has been shown hitherto that NE regulates TNF production and vice versa. Thus, given that cytokine-neurotransmitter interactions influence liver injury and SNS-regulated cytokines modulate both oval cell expansion and liver regeneration, SNS inhibition may promote HPC accumulation and recovery from liver injury indirectly, by effecting cytokine activity.

Theoretically, neurotransmitters may also promote HPC expansion by directly interacting with their receptors on oval cells or their precursors. Such direct effects have been demonstrated for at least one SNS neurotransmnitter, NPY, which interacts with its receptors on neuronal progenitors to induce their proliferation. Although we have shown here that oval cells express apha-1 adrenoceptors, it remains to be seen if their putative bone marrow-derived progenitors also express such receptors. It is tempting to speculate, however, that SNS manipulation might have exerted a direct effect on one or more of the HPC populations, because it has been shown hitherto that the bone marrow receives SNS innervation and adrenoceptors have been demonstrated on certain types of bone marrow derived progenitor cells. Moreover, treatment of mice with PRZ or 6-OHDA has been shown hitherto to mobilize bone marrow-derived hematopoietic progenitors into the circulation, suggesting that injury/inflammation-related increases in NE might normally limit accumulation of HPC. If so, then SNS inhibition would be expected to dis-inhibit this process, permitting expansion of HPC populations within damaged livers. The observation that treatment with PRZ or 6-OHDA increased hepatic accumulation of Thy-1 expressing cells that lack appreciable surface markers for the hematopoietic lineage is consistent with this hypothesis because it has been demonstrated hitherto that such cells can be isolated from the bone marrow of adult rats and induced to differentiate into hepatic oval cells.

Despite these uncertainties about the mechanism(s) through which SNS inhibition promotes HPC expansion, the observation that this process can be-induced by PRZ, a widely available, relatively safe, oral agent, has potential therapeutic implications. In our study, PRZ treatment was well tolerated—none of the PRZ-treated mice died and most developed less cachexia, as well as less liver necrosis and more liver regeneration, than the liver disease controls. These findings complement those of an earlier study which demonstrated that PRZ prevents the development of cirrhosis in carbon tetrachloride-treated rats. Taken together, these results suggest that alpha adrenoceptor blockade might be an effective strategy to arrest liver disease progression.

Recovery from liver damage might be enhanced by encouraging repopulation of the liver by endogenous hepatic progenitor cells. Oval cells are resident hepatic stern cells that promote liver regeneration and repair. Little is known about the mediators that regulate the accumulation of these cells in the liver. Parasympathetic nervous system inhibition reduces the number of oval cells in injured livers. The effect of sympathetic nervous system (SNS) inhibition on oval cell number is not known. Adrenergic inhibition mobilizes hematopoeitic precursors into the circulation and has also been shown to promote liver regeneration. Thus, we hypothesized that SNS inhibition would promote hepatic accumulation of oval cells and reduce liver damage in mice fed anti-oxidant depleted diets to induce liver injury Our results confirm this hypothesis. Compared to control mice that were fed only the anti-oxidant depleted diets, mice fed the same diets with prazosin (PRZ, an alpha-1 adrenoceptor antagonist) or 6-hydroxydopamine (6-OHDA, an agent that induces chemical sympathectomy) had significantly increased numbers of oval cells. Increased oval cell accumulation was accompanied by less hepatic necrosis and steatosis, lower serum aminotransferases, and greater liver and whole body weights. Neither PRZ nor 6-OHDA affected the expression of cytokines, growth factors or growth factor receptors that are known to regulate progenitor cells. In conclusion, stress-related sympathetic activity modulates progenitor cell accumulation in damaged livers and SNS blockade with alpha-adrenoceptor antagonists enhances hepatic progenitor cell accumulation.

The liver's progenitor cell compartment is activated if mature hepatocytes reach a critically low number, such as after severe hepatic injury, or if the mature hepatocytes are prevented from dividing by hepatotoxic drugs. One hepatic progenitor cell (HPC) compartment, the oval cells, is resident within the liver's canals of Herring—the terminal branches of the biliary tree. The source of oval cells is debated. Because transplanted bone marrow cells can rescue experimental animals from liver failure by reconstituting lethally-damaged livers and oval cells express hematopoeitic markers, some have argued that oval cells may be derived from pluripotent progenitors that reside in the bone marrow. It is possible, however, that oval cells are a truly unique population of HPC, and oval cell expression of hematopoetic markers may not be indicative of their lineage. In any case, the factors involved in expanding HPC populations within the liver are not well understood. The identification of such factors is an important goal because they may be useful to support patients with liver failure until a suitable organ is found for transplant. Indeed, if successful, targeted expansion of endogenous HPC may even obviate the need for orthotopic liver transplantation.

The autonomic nervous system (ANS) may regulate the accumulation of HPC in the liver. The parasympathetic nervous system promotes this process because vagotomy reduces HPC in rats with drug-induced hepatitis. Similarly, after transplantation (which surgically denervates the liver), human livers that develop hepatitis have fewer HPC than native, fully innervated livers with similar degrees of injury. This may alter the regenerative response of grafts because the rate of fibrosis is often accelerated in liver transplant recipients with chronic hepatitis.

Although the sympathetic nervous system (SNS) is known to modulate both liver regeneration and hepatic fibrogenesis, it is not known if these effects reflect its ability to influence HPC accumulation. Thus, the present study tests the hypothesis that the SNS regulates the expansion of HPC. We used established models of HPC accumulation involving administration of anti-oxidant depleted diets plus ethionine to cause liver injury and inhibit mature hepatocyte replication. We then inhibited the SNS by adrenoceptor antagonism with prazosin (PRZ) or chemical sympathectomy with 6-hydroxydopamine (6-OHDA) and used flow cytometry and immunohistochemistry to compare HPC numbers in control and SNS-inhibited livers. Because the SNS is known to promote the hepatic accumulation of natural killer T (NK-T) cells, liver NK-T cells were evaluated concurrently to monitor the physiological efficacy of SNS inhibition. Our results demonstrate that SNS inhibition significantly enhances the accumulation of HPC and reduces net liver damage induced by chronic hepatotoxin exposure.

C57BL/6 mice, 10-18 weeks old, from Jackson Laboratory (Bar Harbor, Me.) were subjected to the following experiments.

The diet was a commercial, half-choline deficient diet (ICN, Aurora, Ohio) also 50% deficient in methionine, administered with ethionine (0.15%) in drinking water, to enhance oxidative injury to the liver and cause hepatic accumulation of oval cells within 2 weeks. The combination treatment is hereafter referred to as half methionine choline deficient diet plus ethionine (HMCDE). The control methionine choline diet (CMCD) was also from ICN. Prazosin (PRZ) and DL-Ethionine (E) were from Sigma, St Louis, Mo.).

Chemical sympathectomy was achieved by intra-peritoneal (i.p.) injection of 6-hydroxydopamine (6-OHDA) 100 mg/kg for 5 consecutive days. Thereafter, 6-OHDA was administered at 100 mg/kg i.p., three times per week to ensure continued sympathectomy. This regimen of 6-OHDA treatment depletes norepinephrine in rodent tissues.

Mice were divided into 4 groups (10 to 12 mice/group): Control diet; HMCDE plus saline i.p.; HMCDE plus prazosin in thinking water; HMCDE plus 6-OHDA i.p. Experiments were performed on 2 separate occasions. Therefore, final results are derived from ˜100 mice (10-12 mice/group/experiment×2 experiments).

All mice were weighed at the beginning of the feeding period and weekly thereafter. At sacrifice, sera were collected from all animals and liver tissue from half the animals in each group. These livers were fixed in buffered formalin, preserved in OCT compound (Sakura, Torrance, Calif.) and processed for histology or snap frozen in liquid nitrogen and stored at −80° C. until RNA was isolated. Livers from the remaining animals were prepared for flow cytometry as described below.

Wedges of liver were prepared for histology and immunochemistry as described Anal Biochem 162: 156-159, 1987. Coded, hematoxylin and eosin (H&E)-stained sections were examined by an experienced liver pathologist blinded to treatment groups. Hepatocellular fat accumulation was scored as, no fat=0, focal fat accumulation in <1% of the hepatocytes F, fat in 1-30% of the hepatocytes=1+, fat in 31-60% of the hepatocytes=2+, and fat in 61-100% of the hepatocytes=3+. To evaluate the amount of hepatocyte necrosis, the number of necrotic hepatocytes was counted in 10 randomly selected fields with a 20× lens.

Immunohistochemical analysis of HPC was performed with a mouse monoclonal OV6-type antibody (from Dr Stewart Sell, Albany Medical College, Albany, N.Y.) reacting with cytokeratins 14 and 19; a rabbit polyclonal antibody against 56 and 64 kD human callus cytokeratins (Dako, Denmark) and a rat monoclonal antibody to cytokeratin 19.

Details of the staining procedures are given in Anal Biochem 162: 156-159, 1987. Incubation with the primary antibodies was performed at room temperature for 30 minutes. Mouse monoclonal OV6 antibody and rat anti-cytokeratin 19 were detected using the DAKO Animal Research Kit, peroxidase (Dako, Denmark). The rabbit polyclonal antibody against 56 and 64 kD human callus cytokeratins was detected by anti-rabbit Envision (Dako, Denmark) as described in Carcinogenesis 17: 2143-2151, 1996.

Oval cells were defined as small cells with an oval nucleus and little cytoplasm. These cells occur either singularly or organized in arborizing, ductular structures. They have strong reactivity for liver type cytokeratins, OV-6 and bile duct type cytokeratin 19

To evaluate the effect of treatments on the HPC compartment, coded samples were examined by an experienced liver pathologist blinded to treatment groups. For each liver section, the number of oval cells in 5, randomly selected, non-overlapping, high power (×40 objective) fields was counted. Interlobular bile ducts, were defined as bile ducts with a lumen, associated with a branch of the hepatic artery. Interlobular bile ducts were not considered progenitor cells and, thus not counted as such.

The presence of alpha-1 adrenergic receptors on oval cells was detected on frozen sections using a rabbit polyclonal anti-alpha-1 adrenergic receptor antibody (sc10721, Santa Cruz Biotech, Santa Cruz, Calif., dilution 1/20), followed by undiluted anti-rabbit Envision (Dako, Denmark). For immunofluorescence studies, the anti-alpha-1 adrenergic receptor antibody was combined with a polyclonal antibody against 56 and 64 kDa human callus cytokeratins (Dako, Denmark; dilution 1:100). The primary antibodies were applied sequentially and detected with swine-anti-rabbit FITC or TRITC conjugates. In control sections primary antibodies were omitted. All stainings were performed on 4 representative sections.

Sera were analyzed for alanine aminotransferase (ALT) activity by the Johns Hopkins Clinical Chemistry Laboratory.

Total RNA was isolated from frozen liver samples as described in Mech Dev 120: 117-130, 2003. RNA concentration was determined by optical density and quality assessed by agarose gel electrophoresis and ethidium bromide staining. Ribonuclease protection assay (RPA) kits with probes for murine cytokines (PharMingen, San Diego, Calif.) were used to evaluate factors that might be involved in the recruitment and expansion of HPC after liver injury. The factors studied were Stem Cell Factor (SCF), Hepatocyte Growth Factor (HOF), Interleukin-6 (IL-6), IL.7 IL-11, Leukaemia Inhibitory Factor (LIF), Granulocyte-Macrophage Colony Stimulating Factor (GM-CSF), Macrophage Colony Stimulating Factor (M-CSF), Granulocyte Colony Stimulating Factor (G-CSF), Vascular Endothelial Growth Factor (VEGF), and its receptors, VEGFR1 and VEGFR3.

The hepatic non-parenchymal cell fraction, containing the oval cell and NK-T cell populations, were isolated by described techniques described in Am J Pathol 162: 195-202. 2003 and J Immunol 166: 5749-5754, 2001. Purified mononuclear cells were incubated with noimal mouse serum (Sigma, St Louis, Mo.) and Fc-receptor block (anti-CD16/CD32) to prevent non-specific binding, plus APC-conjugated anti-mouse Thy-1.2 (the C57BL/6 form of the Thy-1 antibody) and antibodies directed against hematopoeitic lineage markers (LIN, a mix of anti-mouse CD4, CD8, CD3, CD45, CD19, Mac-1, Gr-1, Ter119). For NK-T cell labeling, the mononuclear cells were incubated with FITC-conjugated anti-mouse NK-1.1 and PE-conjugated anti-mouse CD3. Antibodies were from Pharmingen acept Ter119—Cedarline lab, Canada. After incubation, washed pellets were fixed with 2% formaldehyde and evaluated by FACS (Becton Dickenson). As described in Hepatology 34: 519-522, 2001, LIN^{-ve}/Thy-1^{+ve} cells, were classified as oval cells. Data was analyzed by Cell Quest software (Becton Dickenson).

All values are expressed as mean E SEM. Group means were compared by unpaired t-test or ANOVA using Graphpad Prism 3.03 (San Diego, Calif.).

To determine the gross effects of the diets, the weights of the animals at the start and end of the experiments were compared. Mice fed the control diet gained a mean of 3 g (12% of starting body weight) during the study (FIG. 1). In contrast, mice fed the HMCDE diet lost a mean of 3 g (12% of starting body weight). Mice fed the HMCDE diet in the presence of PRZ or 6-OHDA, however, only lost a mean of 2 g (7% and 8% of starting body weight). Therefore, SNS inhibition slightly, but significantly, attenuates the weight loss that occurs during consumption of antioxidant-depleted diets.

The treatments also influenced liver mass (FIG. 2a, b). In mice with an intact SNS, as well as in those treated with SNS inhibitors, the HMCDE diet caused an increase in liver mass (FIG. 2a), as well as liver/body mass ratio (FIG. 2b) above that of the control diet. Increases in both parameters tended to be greater in mice treated with SNS inhibitors, but the differences in liver mass achieved statistical significance only for the HMCDE+PRZ treated group. Thus, although SNS inhibition reduced diet-related loss of body mass, it tended to enhance diet-induced hepatomegaly.

Liver histology confirms that, as expected, HMCDE diets caused hepatic steatosis and necrosis (FIG. 3a-c). Histologic evidence of liver injury was accompanied by significant increases in serum ALT values (FIG. 3d), Treatment with 6-OHDA, but not PRZ, significantly reduced the fat score (FIG. 3b). However, both SNS inhibitors significantly reduced hepatic necrosis (FIG. 3c) and serum ALT values (FIG. 3d). These findings demonstrate that PRZ and 6-OHDA-related increases in liver mass occurred despite improvements in hepatic steatosis (6-OHDA) and/or necrosis (PRZ and 6-OHDA). Diet induced liver injury itself elicits the accumulation of oval cells in control mice that were fed the HMCDE diet. The increased HPC were demonstrated immunohistochemically by an increase in the numbers of bile duct type cytokeratin-positive oval cells (FIG. 4a, b) and by flow cytometry quantification of bone marrow lineage marker negative (LIN 8−) cells that expressed Thy 1.2 (FIG. 4c). SNS inhibition with either PRZ or 6-OHDA significantly augments diet-induced oval cell expansion by both assays (FIG. 4a-c). The hepatic accumulation of oval cells is a fairly specific consequence of SNS inhibition because, as expected, the numbers of NK-T cells in the livers of HMCDE-treated mice (8±1% liver mononuclear cells) decrease significantly after treatment with either PRZ (3.5±0.5%, P=0.05) or 6-OHDA (3.6±0.6%, P=0.05). Given that SNS inhibition also reduces HMCDE-induced liver injury (FIG. 3) and stabilizes body weight (FIG. 1), te net effect of SNS inhibition is beneficial in this model of chronic liver injury. Diverse mechanisms may contribute to the hepatoprotective actions of SNS inhibitors.

Other groups have shown that the hepatocyte mitogen, hepatocyte growth factor (HGF), induces oval cell proliferation, promotes liver regeneration and protects the liver from hepatotoxicity. Given the similarities between the effects of SNS inhibition and HGF, it was important to determine if SNS inhibition increased hepatic HGF expression.

Consistent with other reports that liver injury induces compensatory expression of HGF and other factors that promote regeneration, we found that treatment with HMCDE increased the hepatic expression of HGF about 2 fold above control (Table 1). However, SNS inhibition with PRZ or 6-OHDA did not augment this response. Therefore, the hepatoprotective effects of SNS inhibition are not easily explained by HGF induction.

Like certain hematopoeitic progenitor cells, oval cells express c-kit, the receptor for SCF and are responsive to this growth factor. Other cytokines, such as IL-7 and LIF, may also promote progenitor cell accumulation in injured tissues because after cardiac injury, these factors help to recruit bone marrow-derived cells to the injured heart. It has been reported hitherto that IL-6 is expressed by bone-marrow derived cells in regenerating livers and this cytokine has an important hepatoprotective effect because mice that are genetically deficient in IL-6 exhibit inhibited liver regeneration after partial hepatectomy (PH). Other cytokines, such as G-CSF, that signal through gp-130 receptors may be able to compensate for IL-6 deficiency and promote regeneration when the latter cytokine is deficient. VEGF may also play some role in the expansion of HPC because it is a growth factor for hematopoetic stem cells, which express VEGF receptors. To begin to clarify the mechanisms, by which SNS inhibition enhances HPC accumulation in injured livers, we evaluated the effects of SNS inhibition on the heparic expression of G-CSF, GM-CSF, M-CSF, IL-6, IL-7, IL-11, LIF, SCF, VEGF and its receptors VEGFR1 and 3. RPA of whole liver RNA was used to compare the expression of these factors in control (CMCD) mice and mice treated with HMCDE plus or minus SNS inhibitors. No appreciable GM-CSF, M-CSF, IL-6, IL-7, IL-11, SCF or LIF expression could be demonstrated by this assay.

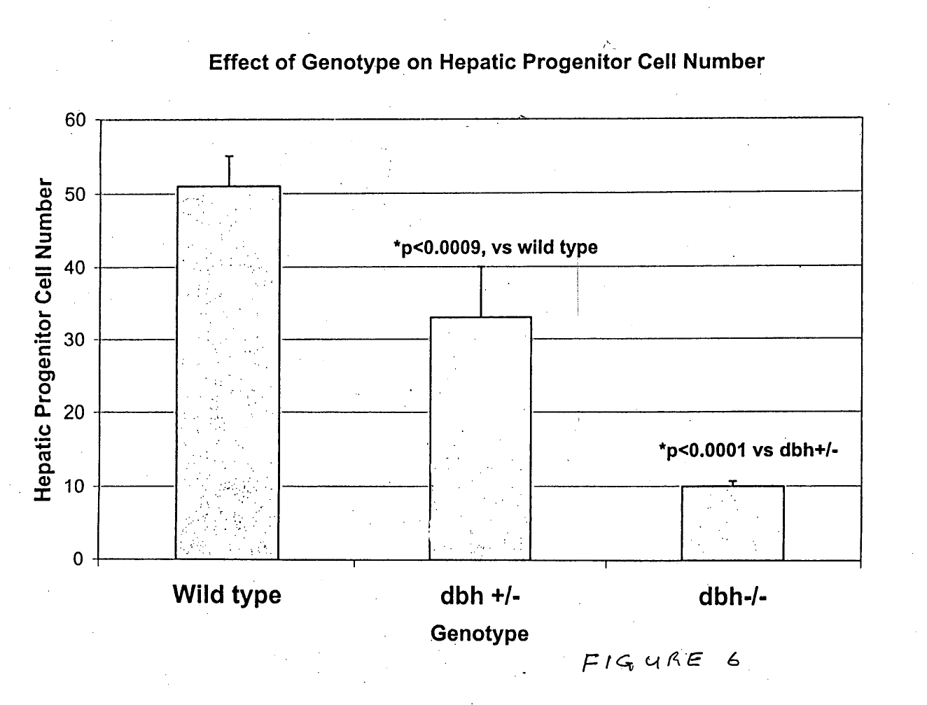

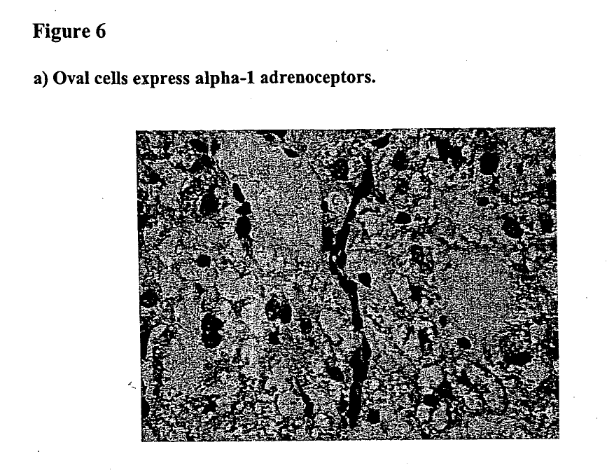

FIG. 6 shows the effect of SNS inhibition on hepatic expression of growth-regulatory factors. Total liver RNA (20 μg per mouse per lane) was evaluated by RPA. Results from 4 mice per treatment group are demonstrated on this representative phospho-image. Similar findings were obtained in a duplicate experiment. Ingestion of the hepatotoxic diet (HMCDE) increased the expression of HGF and G-CSF relative to that of mice fed the control diet (CMCD). These differences are detailed in Table 1.

HMCDE-treatment, however, increased G-CSF expression about 2 fold, regardless of SNS inhibition (Table 1). VEGF and its receptors were expressed in both control and all HMCDE-treated mice, but SNS inhibition did not alter the expression of these factors (Table 1).

TABLE 1

| Treatment | Gene Expression | Statistical Significance |
|---|---|---|
| | HGF | |
| CMCD | 1.0 ± 0.1 | |
| HMCDE | 1.7 ± 0.24 | *P = 0.05, vs CMCD |
| HMCDE + PRZ | 2.1 ± 0.30 | *P = 0.05, vs CMCD; NS vs |
| | | HMCDE |
| HMCDE + 6-OHDA | 2.4 ± 0.75 | *P = 0.05, vs CMCD; NS vs |
| | | HMCDE |
| | G-CSF | |
| CMCD | 0.5 ± 0.04 | |
| HMCDE | 0.8 ± 0.1 | *P = 0.05, vs CMCD |
| HMCDE + PRZ | 1.0 ± 0.3 | *P = 0.05, vs CMCD; NS vs |
| | | HMCDE |
| HMCDE + 6-OHDA | 1.4 ± 0.3 | *P = 0.05, vs CMCD; NS vs |
| | | HMCDE |
| | VEGF | |
| CMCD | 1.7 ± 0.2 | |
| HMCDE | 1.2 ± 0.2 | NS, vs CMCD |
| HMCDE + PRZ | 1.4 ± 0.4 | NS, vs HMCDE |
| HMCDE + 6-OHDA | 2.3 ± 0.5 | NS, vs HMCDE |
| | VEGFR1 | |
| CMCD | 0.3 ± 0.04 | |
| HMCDE | 0.3 ± 0.5 | NS, vs CMCD |
| HMCDE + PRZ | 0.2 ± 0.2 | NS, vs HMCDE |
| HMCDE + 6-OHDA | 0.4 ± 0.1 | NS, vs HMCDE |
| | VEGFR3 | |
| CMCD | 1.1 ± 0.1 | |
| HMCDE | 1.4 ± 0.4 | NS, vs CMCD |
| HMCDE + PRZ | 1.7 ± 0.4 | NS, vs HMCDE |
| HMCDE + 6-OHDA | 2.1 ± 1.0 | NS, vs HMCDE |

NS = Not statistically significant, p > 0.05

In Table 1, Total liver RNA was obtained from 4 mice per treatment group and analyzed by RPA. 20 μg RNA sample from each mouse was evaluated. Results are normalized to concurrently assessed expression of GAPDH in the same RNA samples. Data shown are the mean±SEM results of 4 mice per treatment group. Similar results were obtained in a second experiment.

To determine if the effects of SNS inhibition on the HPC compartment might be mediated via direct interaction between NE and adrenoceptors on HPC, we used immunohistochemistry to determine if oval cells express alpha-1 adrenoceptors. Our results show that bile duct type cytokeratin-positive oval cells do express apha-1 adrenoceptors (FIG. 5a,b). Therefore, direct regulation of HPC by NE is plausible.

Shortages of donor livers for orthotopic liver transplantation have become a major limiting factor in efforts to reduce mortality of patients with end-stage liver disease. Therefore, alternative strategies to replace severely damaged livers must be developed. Studies in mice with massive toxin-induced liver injury have demonsirated that liver cell transplantation can effectively regenerate the liver. Hence, many groups are working to optimize cell transplantation strategies. An alternative, but complementary, approach that might be used to improve the outcome of liver injury involves treatment to encourage repopulation of the liver by endogenous hepatic progenitors. The general feasibility of this strategy is supported by recent evidence that the administration of cytokine mixtures to mobilize native, bone marrow-derived progenitor cells heals experimentally-induced myocardial infarcts in mice. Although transplanted bone marrow cells can also reconstitute lethally-damaged livers, the relative importance of native bone marrow-derived progenitors, or resident hepatic progenitors (i.e. oval cells) and mature hepatocytes, for liver regeneration remains uncertain. Moreover, even if certain progenitor cell populations do contribute to recovery from liver injury, little is known about the mediators that regulate their accumulation within the liver. Therefore, the identification of these factors is an important first step in the development of treatments that seek to expand hepatic progenitor cell populations.

Presumably, endogenously produced factors that induce the hepatic accumulation of liver progenitors are increased, to some extent, during liver damage because this response is evident in most injured livers. However, unless the compensatory increase in proliferative activity of mature hepatocytes or their progenitors can keep pace with liver cell death, recovery is incomplete and damage persists. Therefore, when factors that increase during injury inhibit both mature hepatocyte proliferation and progenitor cell expansion, reconsiruction of a healthy organ becomes compromised. One way to enhance recovery in this situation might be to neutralize the actions of endogenous factors that limit the expansion of native HPC populations. To explore the validity of this concept we studied mice that were treated with HMCDE, because this model of liver injury is known to inhibit replication in mature hepatocytes and increase hepatic oval cells. Our results show that stress-related SNS activity is one of the endogenous factors that limits HPC accumulation in HMCDE-damaged livers, because inhibiting SNS activity magnifies the compensatory expansion of oval cell populations that normally occurs in this model. However, the mechanisms for this remain uncertain.

In rats pre-treated with prazosin immediately before partial hepatectomy AH), the subsequent compensatory induction of hepatocyte DNA synthesis is inhibited. Because liver regeneration after PH results from the replication of mature hepatocytes, this raises the possibility that SNS inhibitors may have compounded the anti-proliferative effects of ethionine and further suppressed mature hepatocyte replication in our model of liver injury. If so, then SNS inhibition might have promoted oval cell accumulation by amplifying poorly-understood signals that trigger expansion of HPC when the replication of mature hepatocytes is inhibited. However, other data argue against this mechanism. For example, the same group who showed that prazosin inhibits hepatocyte DNA synthesis also reported that chronic treatment with SNS inhibitors did not inhibit post-PH liver regeneration in rats. Moreover, Kato and colleagues found that subjecting rats to surgical sympathectomy before PH actually enhanced post-hepatectomy DNA synthesis in the liver. Another group also reported that rats with reduced SNS activity due to ventral median hypothalamic lesions exhibit significantly greater hepatic DNA synthesis at 24 h post-PH and a higher hepatic DNA content from 36 h through 7 days following PH, than sham-operated controls. Thus, the effects of SNS inhibition on the replicative activity of mature hepatocytes appear to be inconsistent. Given this, the massive oval cell expansion that accompanied SNS inhibition in our model may have been mediated by mechanisms other than those that are triggered when the replication of mature hepatocytes is blocked.

As mentioned earlier, liver injury increases the death rate of liver cells and the latter provides a strong stimulus for liver regeneration. We observed many more oval cells in the livers of mice that were treated with SNS-inhibitors, although these groups reproducibly exhibited less severe liver injury than controls, 4 weeks after begining the hepatotoxic diets. We did not study the mice at earlier time points and therefore, cannot directly exclude the possibility that SNS inhibition might have transiently exacerbated diet-induced liver injury, evoking more potent injury-signals to induce compensatory hyperplasia. However, the latter possibility seems very unlikely because it has been reported that liver weight, body weight and liver weight to body weight ratios increase significantly without any associated increase in serum ALT values in healthy rats treated chronically with 6-OHDA to induce chemical sympathectomy. In addition, several groups have demonstrated that NE exacerbates cytokine-mediated hepatotoxicity.

Whereas agents that block NE typically inhibit this and are hepatoprotective. Therefore, it is unlikely that oval cells increased to compensate for an earlier exacerbation of diet-induced liver injury in the mice that received SNS inhibitors.

HGF, IL-6, VEGF and other factors play important roles in liver and other organ regeneration after injury. Because SNS inhibitors enhanced BPC accumulation and improved the outcomes of mice that were exposed to hepatotoxic diets, we expected that SNS inhibitors would increase one or more of these factors, but we were unable to demonstrate this. However, our analysis of whole liver RNA may not have been sufficiently sensitive to detect increased expression of these molecules in small sub-populations of liver cells. Moreover, we cannot exclude the possibility that SNS inhibitors might have sensitized liver cells to the trophic actions of these or other factors. Therefore, whether or not SNS inhibitors interact with other growth factors to enhance hepatic accumulation of oval cells remains an open question.

The latter possibility merits further investigation because it has been reported that TNF-α increases in mice that are fed choline-deficient diets and that proliferating hepatic oval cells produce this cytokine. Moreover, it has been reported that TNF-α is required for oval cell expansion because this response is abrogated by genetic disruption of TNFR1. These observations are particularly intriguing because TNF-α and TNFR1 are necessary for liver regeneration after PH and other types of liver injury. There is strong evidence for cross talk between signaling mechanisms that are activated by TNF-α and those that are modulated by sympathetic neurotransmitters, such as NE. In addition, NE regulates TNF production and vice versa. Thus, SNS inhibition may promote BPC accumulation and decrease liver injury indirectly, by effecting TNF-α activity. We have begun to explore this possibility by comparing hepatic expression of TNF-α mRNA in HMCDE-treated controls and mice treated with HMCDE+PRZ. No differences in TNF-α gene expression were detected in whole liver RNA samples from 3 controls and 3 PRZ-treated mice. However, before firm conclusions can be drawn, these studies must be extended to include more animals and assays for TNF-α protein and activity will be necessary.

Finally, NE may inhibit HPC expansion by directly interacting with its receptors on oval cells or their precursors. Another SNS neurotransmitter, NPY, interacts with its receptors on neuronal progenitors to regulate their proliferation. Although we have shown here that oval cells express alpha-1 adrenoceptors, it is unknown if their precursors also express these receptors. However, the bone marrow receives SNS innervation, adrenoceptors have been demonstrated on certain types of bone marrow progenitor cells, and trealment with PRZ or 6-ORDA mobilizes murine bone marrow-derived hematopoietic progenitors into the circulation. These findings suggest that injury/inflammation-related increases in NE might normally limit accumulation of HPC. If so, then SNS inhibition would be expected to dis-inhibit this process, permitting expansion of HPC populations within damaged livers. The observation that treatment with PRZ or 6-OHDA increased hepatic accumulation of Thy-1 expressing cells that lack appreciable surface markers for the hematopoietic lineage is consistent with this hypothesis.

Controversy rages about the mechanisms that permit hepatic reconstitution of massively damaged livers from bone marrow progenitors, as well as the relative importance of the bone marrow compartment for hepatic regeneration under less extreme circumstances. Our studies were not designed to address either question. Nevertheless, our findings open important new areas for investigation in light of new evidence that donor bone marrow cells can fuse with residual recipient liver cells to generate functional hepatocytes. Bone marrow cells can also differentiate into pancreatic cells. Pancreatic and liver cells are derived from a common progenitor during embryogenesis and in adult rodents, the pancreas may be a source of oval cells. Whether or not SNS inhibition mobilizes bone marrow cells to the pancreas, where they give rise to progenitors that ultimately migrate into the liver and become oval cells, merits further study. Of course, because hepatic oval cells themselves express adrenoceptors, extra-hepatic compartments need not be implicated at all to account for the fact that SNS inhibition increases oval cells in the liver. Adrenoceptor inhibition may directly enhance oval cell survival and more work is also needed to delineate cellular mechanisms that might be involved.

Despite the remaining uncertainties about the mechanism(s) through which SNS inhibition promotes expansion of the endogenous HPC compartment, the observation that this process can be induced by PRZ, a widely available, relatively safe, oral agent, has potential therapeutic implications. In our study, PRZ was well tolerated—none of the PRZ-treated mice died and most developed less cachexia, as well as less liver damage overall than the liver disease controls. These findings complement those of an earlier study which demonstrated that PRZ prevents the development of cirrhosis in carbon tetrachloride-treated rats. Taken together, these results suggest that alpha adrenoceptor blockade might be an effective strategy to reduce the progression of chronic liver disease.

Similar results were obtained in a second experiment.

Little is known about the mediators that regulate hepatic accumulation of oval cells, resident hepatic stem cells. Sympathetic nervous system (SNS) neurotransmitters, e.g., norepinephrine (NE), regulate mature hepatocyte proliferation. Pharmacological manipulation of the SNS also influences oval cell numbers in mice. However, it is not known if oval cells are directly regulated by NE. Therefore, we studied an oval cell line in culture and also determined if oval cells could be increased in the livers of dopamine β-hydroxylase (Dbh)-null mice that are deficient in NE. Similar to mature hepatocytes, cultured oval cells express α1-B and β-2 adrenoceptors, and agonists for these receptors promote oval cell growth in culture. These effects are reduced by α- and β-receptor antagonists, pertussis toxin (a G protein inhibitor) and PD98059 (an ERK pathway inhibitor). NE-deficient Dbh⁺ mice have reduced accumulation of oval cells when treated with methionine/choline deficient, ethionine-supplemented (MCDE) diets that increase oval cell populations in controls. Treating Dbh⁺ with an adrenoceptor agonist during administration of MCDE diets normalizes hepatic oval cell accumulation. Therefore, the SNS neurotransmitter NB is important for hepatic accumulation of oval cells and this process is mediated, at least partially, by direct interaction between NE and oval cell adrenoceptors.

The liver's progenitor cell compartment is activated if mature hepatocytes reach a critically low number, such as after severe hepatic injury, or if the mature hepatocytes are prevented from dividing by hepatotoxic drugs. One hepatic progenitor cell (HPC) compartment, the oval cells, is resident within the liver's canals of Herring—the terminal branches of the biliary tree. Oval cells can differentiate into hepatocytes and cholangiocytes and they express markers of these lineages including cytokeratin (CK)-19. In addition, they express the hematopoietic marker CD-34 and other markers such as OV-6 and the embryonic isoform of pyruvate kinase, M-2-pyruvate kinase. While these markers allow identification of oval cells, the factors regulating the expansion of oval cell populations within the liver are not well understood. The identification of such factors is an important goal because they may be useful to support patients with liver failure until a suitable organ is found for transplant. Indeed, if successful, targeted expansion of endogenous HPC may even obviate the need for orthotopic liver transplantation.

The parasympathetic branch of the autonomic nervous system clearly promotes this process because vagotomy reduces oval cell numbers in rats with drug-induced hepatitis. Similarly, after transplantation (which transects the hepatic branch of the vagus), human livers that develop hepatitis have fewer HPC than native, fully innervated livers with similar degrees of injury. Hepatic oval cells are known to express muscarinic acetylcholine receptors. Therefore, it is possible that parasympathetic neurotransmitters interact directly with these oval cell receptors to regulate the size of the oval cell compartment within the liver.

The sympathetic nervous system also regulates liver regeneration. Mature hepatocytes express adrenoreceptors. Although treatment with catecholamines generally augments mitogen-induced DNA synthesis in cultured hepatocytes, catecholamine-mediated inhibition of G1-S transition has also been reported to occur. Nevertheless, adrenergic agonists are considered to be co-mitogens for mature hepatocytes. We recently identified al-adrenoceptors on hepatic oval cells, suggesting that liver progenitors might also be a target for the SNS during regenerative responses that require oval cell participation. However, it is unclear whether or not hepatocytes and their progenitors (i.e., oval cells) express similar adrenoceptor-classes because the precise adrenoceptor subtypes that are expressed by oval cells is not known. In addition, to our knowledge, no studies evaluating the direct actions of adrenoceptor agonists on oval cell proliferation have been reported. Recently, we showed that al-adrenoceptor antagonism with prazosin (PRZ) or chemical sympathectomy with 6-hydroxydopamine (6-OHDA) increased the numbers of oval cells in the livers of mice treated with a hepatotoxic anti-oxidant depleted diet. The latter observation suggests that SNS neurotransmitters might actually inhibit proliferation of oval cells, as they sometimes do in mature hepatocytes. Differential effects of catecholamines on the proliferation of mature and immature hepatocytes might permit the expansion of the mature cell population while constraining the growth of the other, less mature population. Indeed, differential proliferative responses to growth factors and hormones have already been noted in hepatocytes cultured from fetal, as opposed to adult rat livers. Thus, the aims of the present study are to compare the expression of adrenoceptor subtypes in oval cells and mature hepatocytes, to determine if adrenoceptor agonists directly regulate the growth of oval cells in culture, and to evaluate whether or not oval cell expansion is altered in mice that are genetically deficient in catecholamines.

Mouse hepatic oval cells (HOC) (from Dr. Bryon Petersen, University of Florida College of Medicine, Gainesville, Fla.) were maintained in culture with Iscove's modified DMEM according to the protocol described in PNAS 99: 8078-8083,2002. To confirm that the cells retained their oval cell phenotype in our hands, expression of the embryonic isoform of pyruvate kinase (M2-PK) was evaluated by immunocytochemistry and immunoblot.

Confluent cells were fixed with a 50:50 mixture of cold acetone and methanol and then incubated with pro-block solution (ScyTek, Logan, Utah) to reduce non-specific staining. Samples were subsequently incubated with a goat polyclonal primary antibody to M2-PK (1:2000, Rockland, Gilbertsville, Pa.) an accepted oval cell marker, and/or rabbit polyclonal anti-β2-adrenoceptor (1:200, Santa Cruz Biotech, Santa Cruz, Calif.) for 1 hr at 37° C. followed by donkey anti-goat-Texas red conjugated secondary antibody (1:250, Molecular Probes, Eugene, Oreg.) and/or donkey anti-rabbit-FITC conjugated secondary antibody. Slides were examined with a Zeiss 410 confocal microscope.

Cell homogenates were prepared and protein content was quantified by BSA assay (Pierce, Rockford, Ill.) using bovine serum albumin standards, Proteins (10 μg/lane) were then resolved by polyacrylamide gel electrophoresis and transferred to nylon membranes. After membranes were incubated with primary antibody to M2-PK (1:2000, Rockland, Gilbertsville, Pa.), or α-1_{A}, α-1_{B}, and α-1_{D} adrenoceptors (1:200, Santa Cruz Biotech, Santa Cruz, Calif.), and β1, β2, and β3 adrenoceptors (1:200, Santa Cruz Biotech), peroxidase-conjugated secondary antibodies were added, and antigens were demonstrated by enhanced chemiluminescence (Amersham Biosciences, Piscataway, N.J.) as described in J Biol Chem 277:13037-13044, 2002.

RNA was extracted from oval cells using RNeasy kits (Qiagen, Valencia, Calif.). Concentration and purity were assessed by absorbance at 260/280 nm and then mRNA expression of adrenoceptors was assessed by Rf-PCR analysis. One-step RT-PCR was performed with Superscript one-step RT-PCR with platinum Taq kits (Invitrogen, Carlsbad, Calif.) with Ambion's QuantumRNA Classic II 18S internal standard (Ambion, Austin, Tex.). Products were separated by electrophoresis on a 1.5% agarose gel. Primer sequences and conditions were described in Circulation 105: 380-386, 2002.

Sub-confluent HOC were harvested by gentle trypsinisation and resuspended in serum-free Iscove's modified Dulbecco's minimal essential medium (DMEM, at a density of 5,000 cells/100 μl/well in 96-well plates. Twenty-four hours later, norepinephrine (NE) or isoprenaline (ISO)±various inhibitors—prazosin (10 μM), propranolol (10 μM), pertussis toxin (100 ng/ml), wortmannin (100 nM), SB202190 (10 μM), PD98059 (20 μM), or RO-32-0432 (1 μM) in Iscove's DMEM containing 10a serum were added to some wells, to give a final serum concentration per well of 5%. All drugs were obtained from Calbiochem (San Diego, Calif.) except prazosin and propranolol, which were from Sigmna (St. Louis, Mo.). The inhibitor concentrations used for these studies were similar to those that have been shown to inhibit the growth of other cell types. After 44 hours, cell numbers were assessed by a further 4 h incubation with WST-8 tetrazolium reagent (Dojindo Molecular Technologies, Gaithersburg, Md.) as described in J Clin Invest 106: 501-509, 2000. In viable cells, the tetrazolium salt is metabolized to a calorimetric dye and cell number is proportional to the signal intensity at 450 nm. Therefore, this assay reliably detects treatment-induced changes in cell number.

Hepatocytes were extracted from adult mice by in situ liver perfusion with collagenase as described in Gastroenterology 116: 1184-1193, 1999. RNA was extracted as described in Anal Biochem 162: 156-159, 1987 and then evaluated for adrenoceptor expression using RT-PCR assays described above.

Male Dbh⁺ C57B116 mice and their heterozygous littermates were generated and maintained as previously described in Cell 91: 583-592, 1997, and used at 30-40 weeks of age. Wild type C57B116 mice were from Jackson Laboratory (Bar Harbor, Me.). Animals were allowed access to diets and water ad libitum. To induce oval cell expansion, mice were fed methionine choline deficient diets (ICN, Aurora, Ohio) supplemented with 0.15% Ethionine in the drinking water for 4 weeks. This protocol has been proposed hiterto as an effective strategy for increasing hepatic oval cell numbers in normal C57B1/6 mice. At sacrifice, liver tissues were fixed in buffered formalin or optimal cutting temperature (OCT) fixative (Sakura, Torrance, Calif.) and processed for histology; alternatively, tissues were snap frozen in liquid nitrogen and stored at −80° C. for further analysis.

Immunohistochemical analysis of HPC was performed with a mouse monoclonal OV6-type antibody (from Dr Stewart Sell, Albany Medical College, Albany, N.Y.) reacting with cytokeratins 14 and 19; a rabbit polyclonal antibody against 56 and 64 kD human callus cytokeratins (Dako, Denmark) and a rat monoclonal antibody to cytokeratin 19.

Details of the staining procedures are as described in Am J Pathol 161: 521-530, 2002. Incubation with the primary antibodies was performed at room temperature for 30 minutes. Mouse monoclonal OV6 antibody and rat anti-cytokeratin 19 were detected using the DAKO Animal Research Kit, peroxidase (Dako, Denmark). The rabbit polyclonal antibody against 56 and 64 kD human callus cytokeratins was detected by anti-rabbit Envision (Dako, Denmark) as described in J Pathol 199: 191-200, 2003.

Oval cells were defined as small cells with an oval nucleus and little cytoplasm. These cells occur either singularly or organized in arborizing, ductular structures. They have strong reactivity for liver type cytokeratins, OV-6 and bile duct type cytokeratin 19.

To evaluate the effect of treatments on the HPC compartment, coded samples were examined by an experienced liver pathologist blinded to treatment groups. For each liver section, the number of oval cells in 5, randomly selected, non-overlapping, high power (×40 objective) fields was counted. Interlobular bile ducts, were defined as bile ducts with a lumen, associated with a branch of the hepatic artery. Interlobular bile ducts were not considered progenitor cells and, thus not counted as such.

All values are expressed as mean±SEM. Group means were compared by unpaired t-test using Graphpad Prism 3.03 (San Diego, Calif.).

To confirm that the oval cell line retained its immature phenotype during culture, we evaluated the expression of an accepted oval cell marker, M2-PK. Cultured oval cells uniformly express M2-PK.

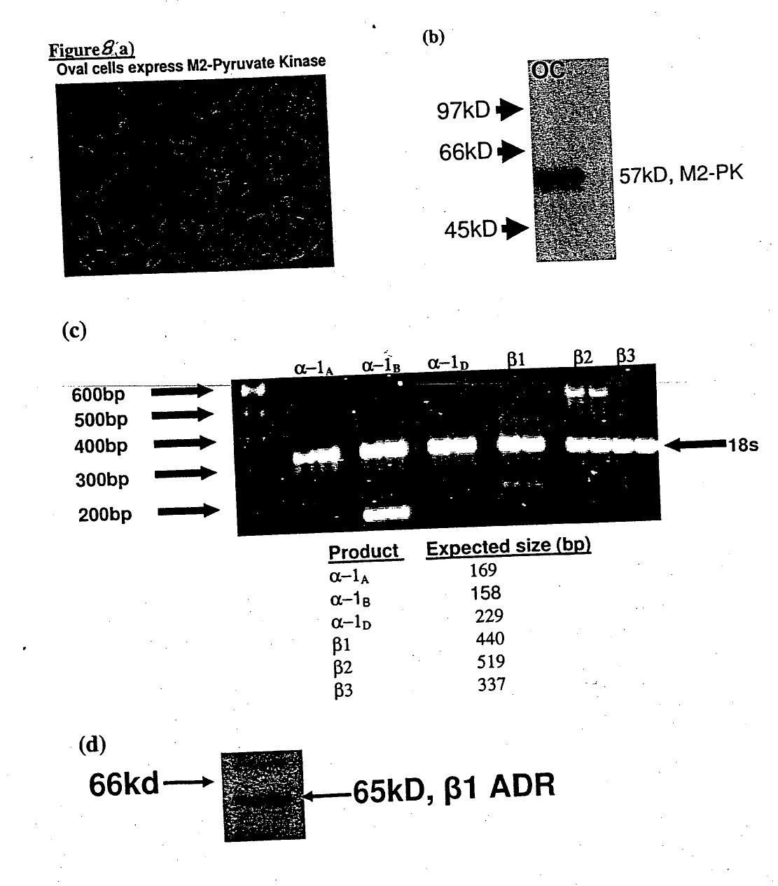

A mouse hepatic oval cell line was evaluated at confluence by immunocytochemistry and immunoblot analysis (10 μg protein/lane) to confirm persistent expression of M2-PK and their immature phenotype. FIGS. 8a-f show the results obtained.

Representative immunocytochemistry (a) and immunoblots (b) are shown. (c) RT-PCR of oval cell RNA was used to analyze the expression of adrenoceptor mRNA Results from a representative analysis of are shown. The first lane shows the DNA ladder (500-200 bp, arrowed). Each subsequent pair of lanes is a replicate analysis of adrenoceptor genes. The 185 band (324 bp) in each lane is shown as a control. (d) Immunoblot analysis (10 μg protein/lane) of oval cell lysates confirms that oval cells express adrenoceptors at the protein level. A representative blot for β1-adrenoceptor is shown. (e) Co-localization of β2-adrenoceptor expression with the M2-PK oval cell marker was demonstrated by immunocytochemistry: Top left panel—M2-PK expression—red; Top right panel—β2-expression—green; Bottom panel—co-ocalization of M2-PK and β2-adrenoceptor expression—yellow. (f) For comparison, RT-PCR was used to analyze the expression of adrenoceptors in mature hepatocytes. Results from a representative analysis of are shown, The first lane shows the DNA ladder (500-200 bp, arrowed). Each subsequent pair of lanes is a replicate analysis of adrenoceptor genes. The 18S band (324 bp) in each lane is shown as a control.

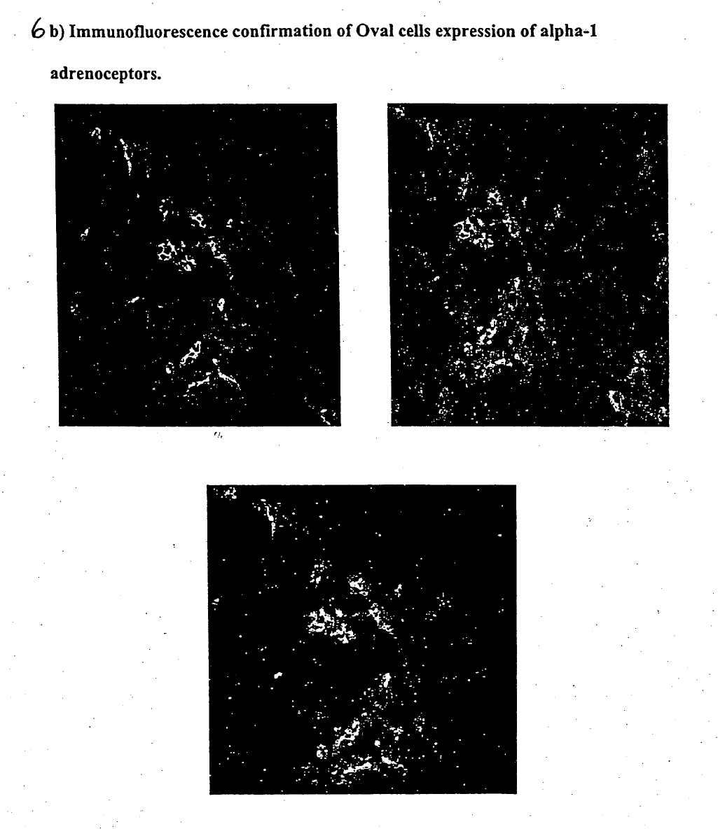

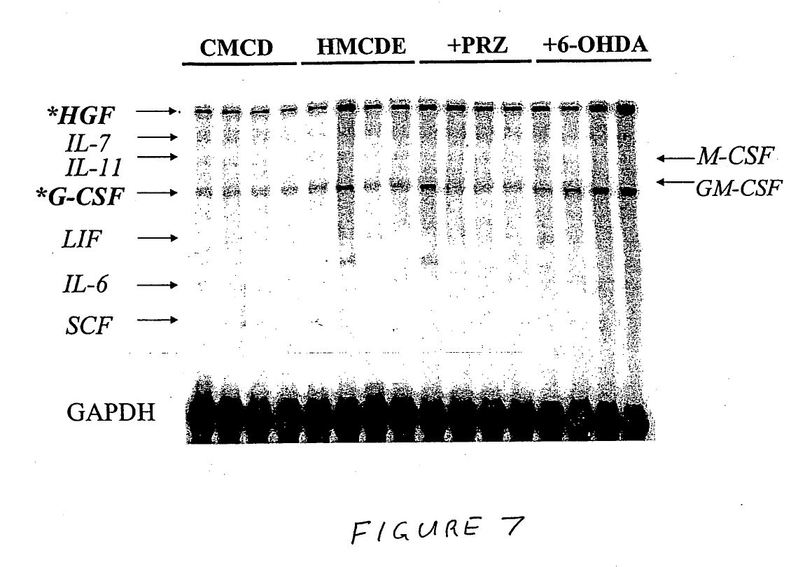

Thus, the culture conditions do not promote oval cell differentiation into mature hepatocytes, which lack this marker. We then used RT-PCR to determine the expression pattern of α1-adrenoceptor and β-adrenoceptor subtypes. Oval cells express predominantly α1-_{B} and β2 adrenoceptors with minor expression of α1-_{D} and β1 (FIG. 8c). There was no detectable expression of α1-_{A}, or β3 adrenoceptors. Immunoblot and immunocytochemnistry analyses revealed a similar pattern of adrenoceptor protein expression. Even adrenoceptors, such as β1 that were weakly-expressed at the mRNA level, were easily demonstrated by immunoblot (FIG. 8d), The predominantly expressed β2-adrenoceptor is well illustrated by immunocytochemistry (FIG. 8e). Next, we used similar techniques to evaluate adrenoceptor subtype expression by mature hepatocytes. Mature primary hepatocytes express α1-B and β2 adrenoceptor mRNA. However, we were unable to demonstrate expression of α1-D or and β1 adrenoceptors (FIG. 7f).

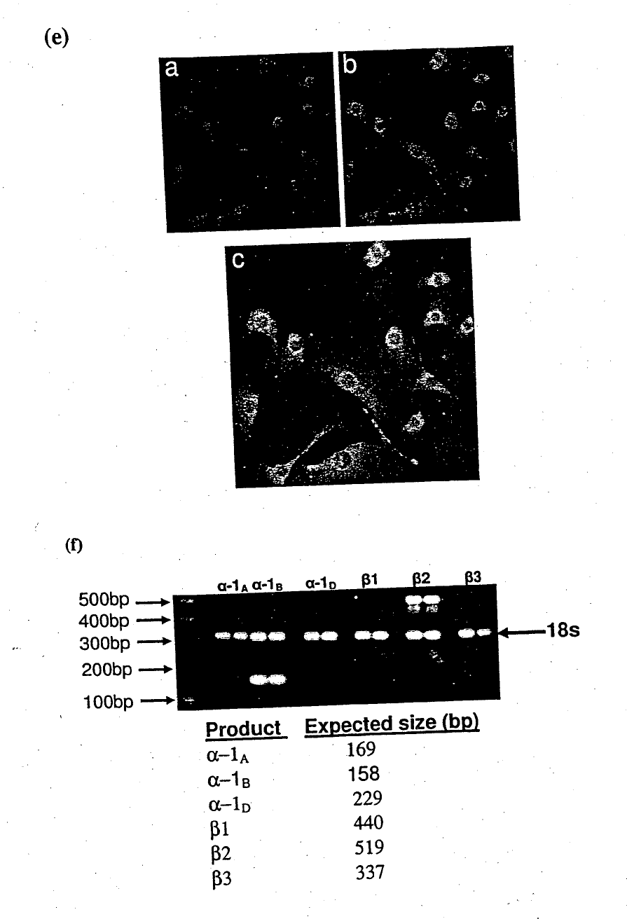

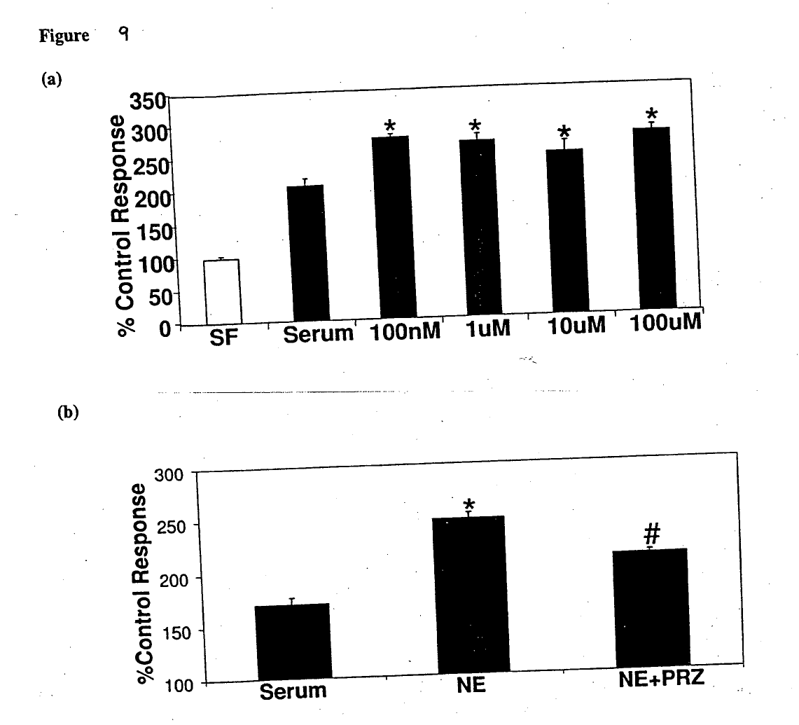

To assess oval cell adrenoceptor function, we incubated oval cells with varying concentrations of NE and ISO, the results being shown in FIGS. 9a-e.

Oval cells were cultured in serum free medium (SF), serum or serum plus increasing concentrations of NE (a) or ISO (c). After 48 hours, the numbers of cells in culture were evaluated. Results are the mean±SD of 2 or more separate determinations. *P<0.05 for 5% serum only versus NE or ISO plus serum. Oval cells were also cultured with NE (100 nM) minus or plus the α₁-adrenoceptor antagonist prazosin (PRZ, 10 μM) (b) or ISO (100 nM) minus or plus the β-adrenoceptor antagonist propranolol (PRL, 10 μM) (d) or the combination of both adrenoceptor agonists minus or plus PRZ (e). Cell numbers were determined after 48 hours. Results are the mean±SD of 2 or more separate determinations. *p<0.05 for serum only versus NE or ISO plus serum; p<0.05 for PRZ vs NE control. PRL vs ISO control and PRZ+PRL vs NE control.

This effect is maximal at 100 nM NE, but persists up to 100 μM NE. NE-induced proliferation is mediated by α-adrenoceptors because it is significantly attenuated by treatment with the α-adrenoceptor antagonist prazosin (FIG. 9b). Similarly, ISO promotes the proliferation of oval cells. The effect appears to be biphasic with peak proliferative activity at 100 nM and 10 mM. The effect of ISO is mediated by β-adrenoceptors because it is attenuated by treatment with the β-adrenoceptor antagonist propranolol (FIG. 9c, d). Although NE and ISO induce oval cell proliferation by interacting with different classes of adrenoceptors, the combination of NE (100 nM)+ISO (100 nM) does not exert an additive effect on oval cell growth in culture (FIG. 8e). Evidence that PRZ (10 μM) blocks catecholamine-induced proliferation under these culture conditions suggests that growth is regulated predominately via ct-adrenoceptors when both α- and β-adrenoceptor agonists are present.

To investigate the post-receptor mechanisms that mediate the actions of NE and ISO on oval cells, we cultured these cells in the presence of specific inhibitors of G-protein, (pertussis toxin); the extracellular signal-regulated kinase (ERK) pathway inhibitor PD98059; the p38 MAP kinase inhibitor, SB202190; the pan-protein kinase C inhibitor, RO-32-0432 and the phosphotidyl-inositol 3-kinase inhibitor, wortmannin. The action of NE and ISO on oval cells are mediated by mechanisms involving G-proteins and ERK because the mitogenic effects of the adrenergic agonists are significantly attenuated by treatment of oval cells with either pertussis toxin (a G-protein inhibitor), or PD98059 (which inhibits MEK, an upstream kinase in the ERK signaling cascade).

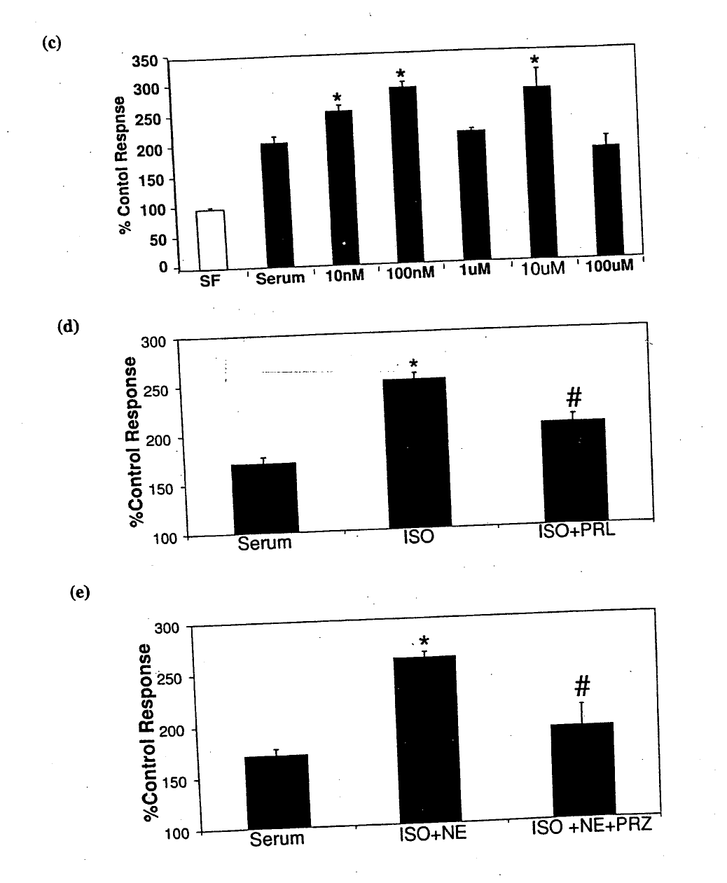

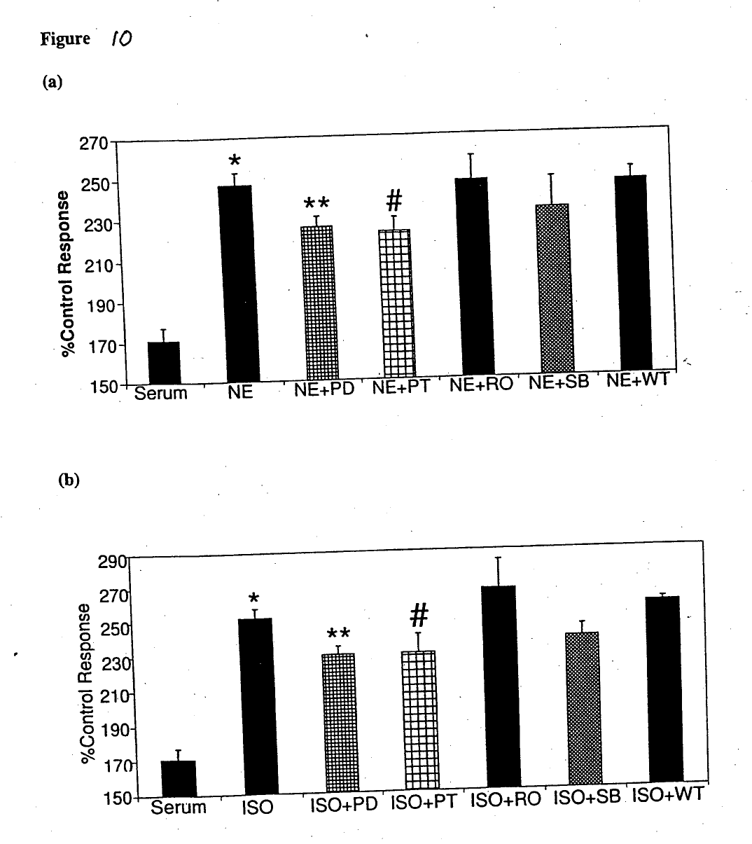

FIGS. 10 a,b show that NE and ISO activate adrenoceptor G protein-coupled mechanisms that induce mitogenic and survival pathways in oval cells. Oval cells culture experiments were repeated with inhibitors of mitogen and/or survival pathways added to some wells. After 48 hours, the numbers of cells in culture were evaluated. PT=pertussis toxin, WI=wortmannin, SB=SB202190, PD=PD98059, RO=RO-32-0432, *P<0.05 for serum only versus NE or ISO plus serum; **P<0.05 for NE or ISO+PD vs NE or ISO alone; #p<0.05 for treated groups versus NE or ISO alone.

FIGS. 10 a,b also shows that treatment with SB202190, a p38 MAPK inhibitor tends to reduce the effect of NE and ISO, but this is not statistically significant.

Because the previous data were acquired by studying an oval cell line in culture, it was necessary to extend our experiments to intact animals to assure that SNS neurotransmitters are truly important regulators of oval cell growth under more physiologically-relevant circumstances. Others have shown that dramatic expansion of hepatic oval cells occurs when normal mice are fed methionine/choline-deficient (MCD) diets supplemented with ethionine in the drinking water for 4 weeks. Therefore, we administered this treatment to dopamine beta hydroxylase Dbh⁺ mice (which have absent biosynthesis of NE and its product, epinephrine, due to targeted disruption of the Dbh gene their heterozygous Dbh⁺ littermates, and wild-type mice. Oval cells are rarely detected in the livers of healthy mice. As expected, MCDE-treatment induces significant oval cell accumulation in wild-type mice, with ˜50 oval cells/high power field observed when liver sections are stained to demonstrate the oval cell marker, OV6. However, MCDE-induced expansion of hepatic oval cells is reduced by about 40% in Dbh⁺ mice, and even more suppressed in Dbh⁺ mice which exhibit only 10 oval cell/HPF after 4 weeks of MCDE treatment.

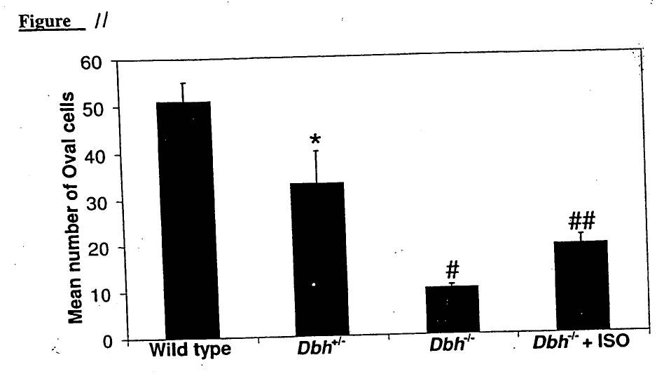

Reduced numbers of oval cells in NE-deficient Dbh⁺ mice were observed, the results being shown in FIG. 11.

Dbh⁺ and their control Dbh⁺ littermates were fed methionine choline deficient (MCD) diets to induce oval cell expansion. A subgroup of the Dbh⁺ mice was also infused with isoprenaline (ISO). After 4 weeks, liver samples were obtained, fixed in formalin and paraffin-embedded. Oval cell numbers were counted in 5 randomly selected fields/section from 4 mice/group. Mean±SD results of one experiment are graphed. Virtually identical results were obtained in a second experiment that studied an additional 4 mice/group. *p<0.05 for Dbh⁺ versus wildtype, #p<0.05 Dbh⁺ versus Dbh⁺ and ##p<0.05 Dbh⁺+ISO versus Dbh⁺.

In the present study we have shwn that ovel cells are regulated by SNS neurotransmitters. This process is likely to be mediated, at least in part, via direct interaction between the catecholainines and adrenoreceptors because oval cells express multiple adrenoreceptor subtypes—predominantly α-1B and β2, but also α-1D and β1. Moreover, these oval cell adrenoreceptors are functional, as demonstrated by evidence that α- and β-adrenoreceptor agonists (e.g., NE and ISO) significantly promote the proliferation of cultured oval cells, and this effect is attenuated by the adrenoreceptor antagonists, PRZ and PRL. The mitogenic effects of NE and ISO are inhibited by treating cultured oval cells with pertussis toxin and PD98059, suggesting that G-proteins and ERK kinases transduce some of the groth-promoting signals initiated by oval cell adrenoreceptors. Finally, studies in mice support the physiological improtance of th aforementioned mechanisms in regulating hepatic oval cell populations. Dbh⁺ mice, which are geneticall defficient in NE and its product, epinephrine, exhibit inhibited hepatic accumulation of oval cells when treated with agents that dramatically increase oval cell inducers restores oval cell expansion in the Dbh⁺ group, providing that adrenoreceptor activation plays an important role in the hepatic accumulation of oval cells that occurs in response to these oval cell inducing agents.

On the other hand, evidence that reduced adrenoreceptor activity limits oval cell accumluation in Dbh⁺ mice is difficult to reconcile with our recent findings in normal mice. When the latter are fed MCD diets supplemented with ethionine, treatment with PRZ (to block alpha adrenoreceptors) or 6-hydroxydopamine (to induce chemical symnpathectomy) dramatically amplifies the expansion of hepatic oval cell populations (REF), suggesting that adrenoreceptor activity normally suppresses the growth of hepatic oval cells. The contradictory findings of our two studies might be explained by differential effects of adrenoreceptor agonists on oval cells and their progenitors. The present oval cell culture data clearly demonstrate that direct activation of oval cell adrenoreceptors promotes oval cell growth. There is some, albeit hotly debated evidence that oval cells may be derived from bone marrow progenitors. Inhibition of SNS activity by PRZ or 6-hydroxydopamine is known to mobilize hematopoeitic progenitors from bone marrow. Thus, decreases in adrenoceptor function may facilitate the release of oval cell progenitors from the bone marrow, while the present findings suggest that increased adrenoceptor activity may enhance growth of more mature oval cells within the liver. Other mechanisms may also be involved in the Dbh-deficient mice, because these animals have altered levels of other neurotransmitters, such as dopamine and neuropeptide Y, and some of these factors are known to regulate stem cell viability. In addition, as discussed below, catecholamines influence the production and activities of other factors, including cytokines and chemokine receptors, that modulate the homing, engraftment and survival of progenitor cells within the liver.

Although the field of liver stem cell research is still in its infancy, researchers are beginning to identify factors that regulate hepatic progenitors. Unfortunately, however, the published literature contains relatively little information about the intracellular signals that these factors evoke in any given hepatic progenitor cell population. In addition, almost nothing has been reported yet about how different factors might interact to modulate the growth and differentiation of either bone marrow-derived or resident hepatic progenitors. Oval cells, progenitor cells that reside in the livers of adult organisms, have been studied far more extensively than their putative, bone marrow-derived precursor.

Until now, attention has focused predominately on the role of injury-related cytokines and chemokines as regulators of hepatic oval cell populations. For example, it is known that oval cells are capable of producing tumor necrosis factor (TNF)α. This cytokine promotes hepatic oval cell accumulation because mice with targeted disruption of the TNF receptor-1 (TNFR-1) gene cannot increase hepatic oval cells in response to treatment with MCD diets+ethionine. The latter observation is intriguing because proliferative responses of mature hepatocytes are also inhibited in TNFR-1-deficient mice and inhibited replication of mature hepatocytes is generally thought to stimulate expansion of hepatic oval cell populations. Whether or not TNF-α, or TNFα-induced cytokines such as interleukin (IL)-6, directly regulate the viability and/or proliferation of oval cells themselves has not been evaluated. Pertinent to our findings, catecholamines can increase both TNF-α and IL-6 in some circumstances. However, in an earlier study, we were unable to demonstrate any change in hepatic expression of either cytokine following experimental manipulation of SNS activity in MCD diet-fed mice that had been treated with ethionine

Stromal derived factor (SDF)-1a, an important chemotactic and viability factor for both neuronal and hematopoeitic progenetors, may also regulate hepatic oval cells because these cells express CXCR4, the receptor for SDPF-1a, and migrate along a SDF-1a gradient during in vitro chemotaxis assays. In massively injured livers where oval cells participate in the regenerative response, rat hepatocytes up-regulate expression of SDF-1a, prompting speculation that SDF-1a/CXCR4 interactions are involved in expanding oval cell populations during some types of liver injury. Hepatic accumulation of CXCR4+ cells has also been noted in injured human livers in which bile duct epitheial cells express SDF-1a. Moreover, in another recent study of NOD/SCW mice, neutralization of CXCR4 abolished homing and engraftment of the murine liver by human CD34+ hematopoeitic progenitors. In the NOD/SCID hosts, injection of human SDF-1 also increased homing of the bone marrow-derived progenitors, which subsequently differentiated into albumin-producing cells that were localized in clusters surrounding bile ducts

To our knowledge, the role of SDF-1a and/or CXCR4 in enhancing hepatic accumulation of oval cells in mice fed MCD diets supplemented with ethionine has not been evaluated. However, given the apparent importance of SDF1a/CXCR4 in other types of liver injury, this certainly merits investigation in the future. As mentioned earlier, TNF-α is necessary for oval cell expansion in mice treated with MCD diets plus ethionine. There are reports that TNF-α and TNFα-induced cytokines induce CXCR4 expression, but inhibit production of SDF1-a. On the other hand, SDP1-a increases TNFα production by some CXCR4-expressing cells. Others have shown that hepatocyte growth factor (HGF) up-regulates CXCR4 expression and enhances SDF-1 mediated chemotaxis by CD34+ bone marrow progenitors. We recently reported that hepatic HGF expression is increased significantly in mice that have been treated with MCD diets plus ethionine. Given this background, it would not be surprising if CXCR4-expressing cells accumulate in the livers of mice during treatment with MCD diets and ethionine. Manipulation of SNS activity did not alter hepatic TNF alpha or HGF expression in earlier studies.

Nevertheless, changes in the relative abundance of adrenergic agonists might modulate the signaling of CXCR4 receptors in cells that express both adrenoceptors and CXCR4, because all of these receptors couple to G proteins and G proteins transduce SDF-1a/CXCR4-initiated survival signals in other cells. Interestingly, there is also some suggestive evidence that NE itself might up-regulate CXCR4 expression in some cell types. Finally, as mentioned earlier, agents that inhibit SNS activity enhance the release of hematopoeitic progenitors from the bone marrow, and it was recently proven that CXCR4 function must be inhibited in order to mobilize bone marrow-derived stem cells. Thus, it is tempting to speculate that interactions between catecholamines, cytokines and chemokines may modulate CXCR4 function and thereby, alter hepatic oval cell populations. Much work will be necessary to evaluate this possibility carefully. In any case, the present data extends our earlier work with SNS inhibitors and provides additional evidence that SNS neurotransmitters are capable of acting at multiple levels to regulate oval cell accumulation in injured livers. As such, this information identifies the SNS as a potential target for therapeutic manipulation to regulate expansion of this progenitor cell population in injured livers.

1. A method of treating diseased or damaged tissue which comprises administering an agent for mobilizing stem cells.

2. A method according to claim 1, wherein the diseased or damaged tissue is the liver.

3. A method according to claim 1, wherein the agent comprises at least one regulator of the sympathetic nervous system.

4. A method according to claim 3, wherein said regulator comprises an adrenosceptor agonist.

5. A method according to claim 3, wherein said regulator comprises an adrenoceptor antagonist.

6. A method according to claim 5, wherein said regulator comprises an α-adrenoceptor antagonist or a β-adrenoceptor antagonist.

7. A method according to claim 6, wherein said regulator comprises an α-1_{B}-adrenoceptor antagonist for hepatic oval cells.

8. A method method according to claim 6, wherein said regulator comprises an β20-adrenoceptor antagonist for hepatic oval cells.

9. A method according to claim 6, wherein the regulator comprises prazosin.

10. A method according to claim 6, wherein the regulagtor comprises propanalol.

11. Use of an agent for the mobilization of stem cells in the manufacture of a medicament for the treatment of diseased or damaged tissue.

12. Use according to claim 11, wherein the agent comprises an adrenoceptor agonist.

13. Use according to claim 11, wherein the agent comprises an adrenoceptor antagonist.

14. Use according to claim 13, wherein the agent comprises an α-adrenoceptor antagonist or a β-adrenoceptor antagonist

15. Use according to claim 14, wherein the agent comprises an α-1_{B}-adrenoceptor antagonist for hepatic oval cells.

16. Use according to claim 1r4, wherein the agent comprises an β2-adrenoceptor antagonist for hepatic oval cells.

17. Use according to claim 14, wherein the regulator comprises prazosin.

18. Use according to claim 14, wherein the regulator comprises propanalol.
